(19) United States
(12) Patent Application Publication
(10) Pub. No.: US 2008/0206334 A1
(43) Pub. Date: Aug. 28, 2008

(54) Pharmaceutical Polymer Composition For Oral Controlled-Release Delivery Of Terbutaline Sulfate

(76) Inventors: Mayyas Al-Remawi, Russiefa (JO); Adnan Badwan, Amman (JO)

Correspondence Address: SCHMEISER, OLSEN & WATTS, 22 CENTURY HILL DRIVE, SUITE 302, LATHAM, NY, 12110, US

(21) Appl. No.: 11/813,301

(22) PCT Filed: Dec. 29, 2005
(86) PCT No.: PCT/EP05/13530

§ 371 Date: Jul. 3, 2007

(30) Foreign Application Priority Data
Jan. 19, 2005 (EP) 05000985.1

(51) Int. Cl. A61K 9/26; A61K 31/138; A61K 9/00

(52) U.S. Cl. 424/469; 514/654; 424/485

(57) ABSTRACT

The present invention is directed to a controlled-release pharmaceutical composition providing a sustained delivery of the basic drug Terbutaline sulfate, said composition comprising at least Terbutaline sulfate or a derivative thereof as an active agent, and further comprising an inactive matrix, said matrix comprising a hydrophilic polysaccharide polymer mixture, said mixture comprising chitosan or a derivative thereof, and further comprising xanthan gum or a derivative thereof, wherein the ratio of xanthan gum and chitosan within said mixture is in the range from about 1:10 to about 10:1, and said composition optionally comprising sodium bicarbonate.

The present invention is directed to a controlled-release pharmaceutical composition providing a sustained delivery of the basic drug Terbutaline sulfate, said composition comprising a hydrophilic polysaccharide polymer mixture of chitosan and xanthan gum and further comprising sodium bicarbonate.

### BACKGROUND OF THE INVENTION

Polymers that hydrate in an aqueous medium and form a gel structure are known as hydrophilic polymers such as chitosan and xanthan gum. These polymers are either cationic (as chitosan) or anionic (as xanthan) in chemical nature. Binary mixture of these polymers will lead to the formation of a gel layer at the surface of the tablet resulting in slow release of the active material out of the tablet. As a consequence, a prolongation of drug release is achieved.

When a solid dosage form comes into contact with acidic medium, as found in the stomach, for example, the hydratable polymers swell to form a gel. Sodium bicarbonate acts as a buffering agent that keeps the tablet micro-environment at pH 6-7. A cross-linking reaction occurs between cationic chitosan and anionic xanthan gum at this pH range. Thus, a layer of complex coacervate (hydrogel layer) is formed at the surface of the tablet. The drug to be released out of the tablet has to cross this hydrogel layer. The hydrogel barrier slows down the drug flux out of the tablet resulting in release retardation. Another mechanism for drug release retardation is provided by sodium bicarbonate. Sodium bicarbonate releases carbon dioxide (CO₂) gas inside the gel layer. The tablet density becomes low and a floating tablet is formed. CO₂ bubbles attached to the gel act as a barrier for drug release. Another advantage of CO₂ gas formation is the generation of a buoyant system. This system increases tablet resident time inside the gastrointestinal tract.

As described earlier in the European patent application No. 03019532.5, a ratio of 1:1 of chitosan and xanthan gum has been found a suitable universal controlled-release composition for controlling the release of basic drugs with a drug to polymer mixture ratio of 1:3 to 1:10.

Terbutaline sulfate is one example of a water-soluble basic drug. The solubility in water is >20 mg/ml, and the dissociation constant (pKₐ) values have been reported to be 8.8, 10.1 and 11.2. The value of pKₐ=10.1 can be assigned to the amino group. The other two pKₐ values, 8.8 and 11.2, may be attributed to the aromatic hydroxyl groups (S. Ahuja and J. Ashman, Terbutaline Sulfate. In: Analytical Profiles of Drug Substances, K. Florey (ed.), Vol. 19, Academic Press, 1990, pp. 603-625). Herein, Terbutaline sulfate was selected as a model drug.

Terbutaline sulfate is a beta-2 adrenergic agonist that is used as a bronchodilator. By relaxing bronchial tubes, Terbutaline prevents and treat wheezing, shortness of breath, and troubled breathing caused by asthma. It also is thought to relax the uterus' muscles, and thus, its use in preterm labor has been suggested. Terbutaline sulfate is typically prescribed in the pill form (2.5 or 5 mg pills at 3- 4- or 6-hour intervals), is administered by subcutaneous injection (e.g. by a pump with dosages of at least 3 mg per 24-hours) or is received by aerosol inhalation. The latter administration route takes the quickest therapeutic effect. However, in some indications it is preferred that the drug is delivered more steadily. When administered orally, Terbutaline sulfate has a rather short biological half-life (about 3.6 hrs). To overcome this disadvantage, its formulation in a controlled-release system would be of advantage (S. Ahuja and J. Ashman, supra).

Therefore, it is an object of the present invention to provide for a controlled-release pharmaceutical composition formulated for oral controlled-release delivery of the basic drug Terbutaline sulfate.

### SUMMARY OF THE INVENTION

The object of the present invention is solved by a controlled-release pharmaceutical composition comprising at least Terbutaline sulfate or a derivative thereof as an active agent, and further comprising an inactive matrix, said matrix comprising a hydrophilic polysaccharide polymer mixture, said mixture comprising chitosan or a derivative thereof, and further comprising xanthan gum or a derivative thereof, wherein the ratio of xanthan gum and chitosan within said mixture is in the range from about 1:10 to about 10:1.

In one embodiment, the ratio of xanthan gum and chitosan is in the range from about 1:2 to about 2:1.

In a preferred embodiment, the ratio of xanthan gum and chitosan is 1:1.

In one embodiment, the ratio of Terbutaline sulfate and the hydrophilic polysaccharide polymer mixture is in the range from about 1:1 to about 1:5.

In a preferred embodiment, the ratio of Terbutaline sulfate and the hydrophilic polysaccharide polymer mixture is 1:3.

In one embodiment, the controlled-release pharmaceutical composition optionally comprises at least one additional pharmaceutically acceptable filler.

In a preferred embodiment, the pharmaceutically acceptable filler is selected from the group comprising calcium hydrogen phosphate, mannitol, Avicel PH 101, sodium bicarbonate and the like.

In another preferred embodiment, the pharmaceutically acceptable filler is a carbon dioxide generating and/or pH controlling filler.

In a particular preferred embodiment, the pharmaceutically acceptable filler is sodium bicarbonate.

In one embodiment, the percentage of the pharmaceutically acceptable filler is in the range from about 5% to about 20% by total weight.

In a preferred embodiment, the percentage of the pharmaceutically acceptable filler is 12.5% by total weight.

In one embodiment, the percentage of the pharmaceutically acceptable filler together with the hydrophilic polysaccharide polymer mixture is in the range from about 70 to about 90% by total weight.

In a preferred embodiment, the pharmaceutically acceptable filler together with the hydrophilic polysaccharide polymer mixture is 83% by total weight.

In one embodiment, the ratio of Terbutaline sulfate and the pharmaceutically acceptable filler on the one hand and the hydrophilic polysaccharide mixture on the other hand is in the range of about 1:1 to about 1:5.

In a preferred embodiment, the ratio of Terbutaline sulfate and the pharmaceutically acceptable filler on the one hand and the hydrophilic polysaccharide mixture on the other hand is 1:5.

In one embodiment, a single dosage unit of Terbutaline sulfate is about 4 to 8% by total weight.

The object of the present invention is further solved by a method for preparation of a controlled-release pharmaceutical composition comprising at least Terbutaline sulfate or a derivative thereof as an active agent, said method comprising the following steps:
(a) preparing a mixture comprising chitosan or a derivative thereof, xanthan gum or a derivative thereof, Terbutaline sulfate or a derivative thereof and optionally at least one additional pharmaceutically acceptable filler;
(b) compacting said mixture, preferably into a tablet.

In one embodiment, the tablet comprises from about 5 mg to about 7.5 mg Terbutaline sulfate.

In a preferred embodiment, the tablet comprises 5 mg Terbutaline sulfate.

The object of the present invention is further solved by a use of a controlled-release pharmaceutical composition comprising at least Terbutaline sulfate or a derivative thereof as an active agent for the manufacture of a medicament for inducing muscle relaxation in an animal, preferably a mammal, and most preferably human.

The object of the present invention is further solved by a method of treating illness of chronic nature in need of such treatment which comprises administering to the patient a therapeutically active agent that is basic in chemical nature or a salt of basic drug such as Terbutaline sulfate, the formula of prolonged release delivery contains xanthan gum and chitosan as a release retarding polymer carrier mixture and sodium bicarbonate as a buffering agent.

In one embodiment, the ratio of xanthan gum (anionic polymer) to chitosan (cationic polymer) 1:1 part by weight.

In one embodiment, the ratio of active agent and buffering agent (Terbutaline sulfate and sodium bicarbonate) to polymer carrier mixture (chitosan and xanthan gum) ratio is 1:5.

In one embodiment, the controlled-release carrier consists essentially of a binary polymer mixture of chitosan and xanthan gum and sodium bicarbonate as a buffering system and release modifier as a direct compression formula.

In one embodiment, the tablet contains 5-7.5 mg Terbutaline sulfate.

In one embodiment, the controlled-release carrier is present in the range of 70-90%, specifically 83% by weight of the formulation.

In one embodiment, the controlled-release carrier is in the ratio of 1:1 by weight.

In one embodiment, the buffer system is a carbon dioxide generating and pH controlling system such as sodium bicarbonate and basic excipient that has a similar action.

In one embodiment, the buffer system is in the range of 5-20%, specifically 12.5% by weight.

The object of the present invention is further solved by a method of administrating Terbutaline sulfate or any basic drug and salt of a basic drug thereof in a controlled release dosage form to a patient in need thereof, said method comprising administration of solid unit dose of a therapeutic active agent of Terbutaline sulfate thereof and controlled-release carrier of xanthan gum and chitosan to an extent of at least 70% by weight of said carrier, wherein the controlled release carrier is present in a ratio of 1:1 parts by weight, the ratio of Terbutaline sulfate or any basic drug or salt of basic drug and the buffer system sodium bicarbonate to the controlled release carrier is 1:5.

In one embodiment, the controlled release solid dosage form is preferably formulated in a readily flowable direct compressible simple formula. However, granulation of such formula found not to affect its controlled release behavior.

In one embodiment, the controlled-release dosage form contains Terbutaline sulfate in the range of 4-8% by weight.

In one embodiment, the controlled-release solid dosage form is more stable under accelerated stability conditions in comparison with a commercial controlled-release product.

In one embodiment, the controlled release solid dosage form is obtained by simple mixing of the formula components and directly compress them using a suitable compaction machine.

The term “controlled-release” as used in the context of the present invention refers to a temporal and/or spatial control. “Temporal” can indicate a “sustained release” or any release altered or modulated with respect to time. Preferably, “controlled-release” refers to a “sustained release” or “prolonged release” or “release retardation”. “Spatial” refers to any localized delivery.

An “active agent” in this context generally means a pharmacologically, therapeutically or otherwise effect agent which may have an effect itself or may become active, e.g. after being metabolized by endogenous enzymes or being converted under certain in vivo reaction conditions (e.g. at a certain pH).

According to the present invention, Terbutaline sulfate may be employed as any pharmaceutically acceptable salt thereof. Derivatives of Terbutaline are also considered.

An “inactive matrix” in this context means those components of the controlled-release pharmaceutical composition which serve as a carrier for the active agent without being itself pharmacologically, therapeutically or otherwise effective. Preferably, the active agent becomes mixed with the inactive matrix components during preparation of the controlled-release pharmaceutical composition such that the active agent is dispersed or otherwise embedded within the inactive matrix.

Apart from chitosan, also other hydrophilic polysaccharide polymers belonging to the group of chitin and salts and derivatives thereof may be considered.

When the term “% by weight” is used to indicate a percentage, this refers to the compostion's total weight, if not otherwise indicated.

A “ratio” as used herein generally refers to a ratio by weight, i.e. “w/w” and “w:w”, respectively, if not otherwise indicated.

Preferably, controlled-release pharmaceutical compositions of the present invention are intended for the preparation of tablets generated by compaction, and preferably by direct compaction. The term “direct compaction” or “direct compression” means that all components constituting the controlled-release pharmaceutical composition are geometrically mixed before compression. Thus, compaction can be performed with powder. However, any processing, e.g. preparation of granules, which granules are subjected to compression, is also considered by the present invention. Furthermore, the use of the controlled-release pharmaceutical composition of the present invention for preparing other solid dosage forms, e.g. capsules, is also considered. Also taken into consideration is any refinement of the solid dosage form, e.g. coating of tablets. The controlled-release pharmaceutical composition of the present invention may also be incorporated within further delivery devices, e.g. a controlled-release plaster or a diaphragm pessary.

The capability of “pH controlling” means that a compound is able to maintain a certain pH, i.e. is a buffering agent.

One example of a “basic excipient” is sodium bicarbonate, which is used herein as a release modifying excipient.

A “controlled-release carrier” is a “controlled-release matrix”.

In conclusion, the present invention provides for a controlled-release pharmaceutical composition having superior properties with respect to Terbutaline sulfate delivery compared to controlled-release compositions belonging to the state of the art.

### DETAILED DESCRIPTION OF THE INVENTION

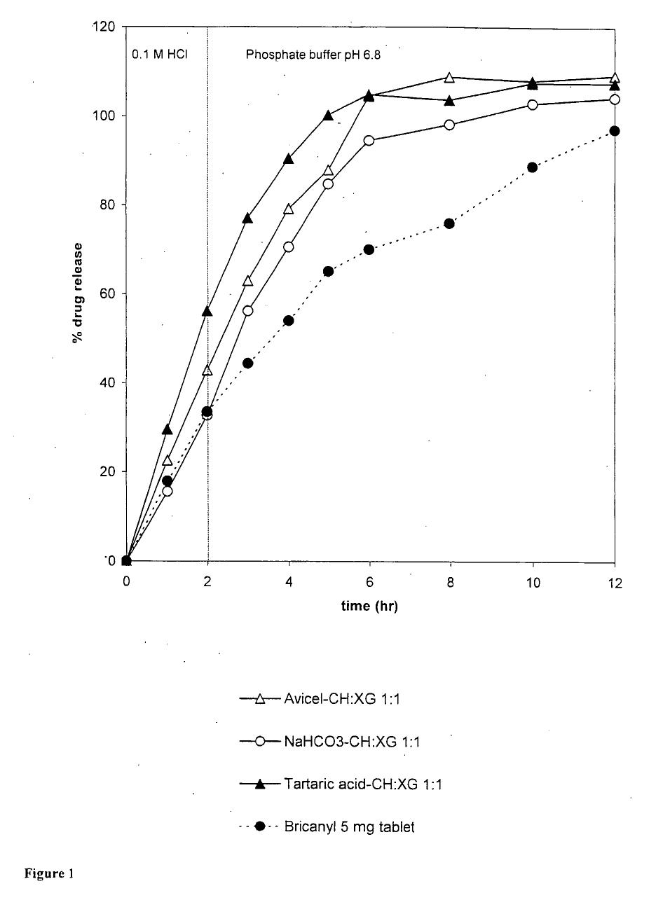

The invention shall now be further described and illustrated by the following examples with making reference to the attached FIGS. 1-29. All examples are provided by way of example only and are not intended to limit of the scope of the invention.

FIG. 1 shows the time course of Terbutaline sulfate release from a controlled-release matrix of (CH:XG 1:1) comprising different excipients (D/E:P 1:3) compared to a Bricanyl 5 mg tablet.

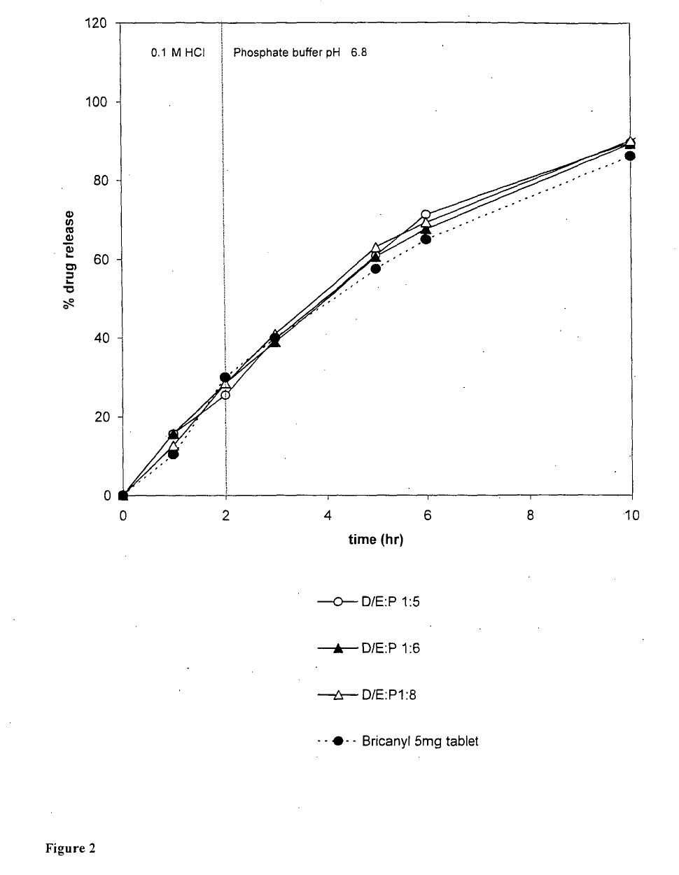

FIG. 2 shows the time course of Terbutaline sulfate release from a controlled-release matrix of (CH:XG 1:1) comprising NaHCO₃ as a release modifying excipient at different D/E:P ratios compared to a Bricanyl 5 mg tablet.

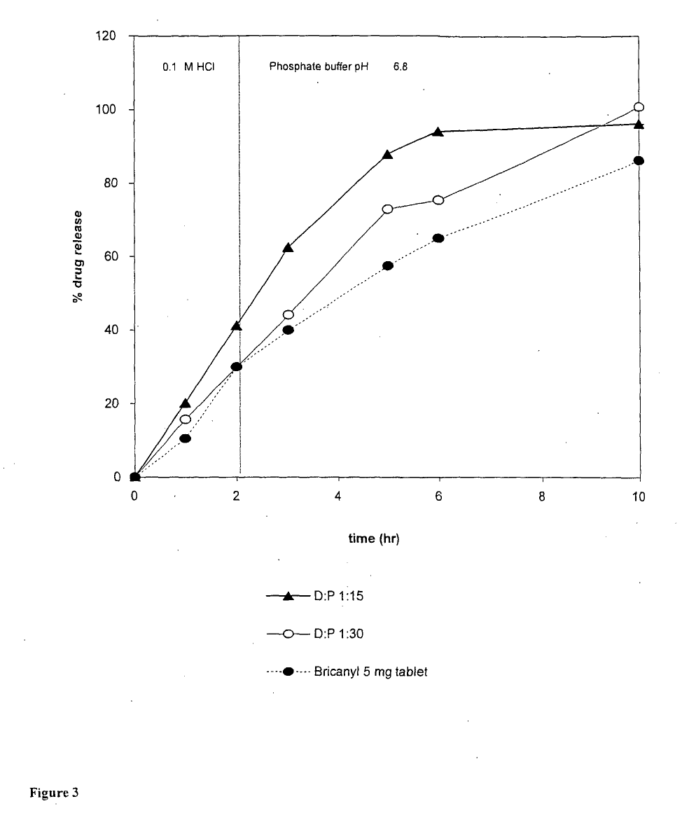

FIG. 3 shows the time course of Terbutaline sulfate release from a controlled-release matrix of (CH:XG 1:1) at different D:P ratios compared to a Bricanyl 5 mg tablet.

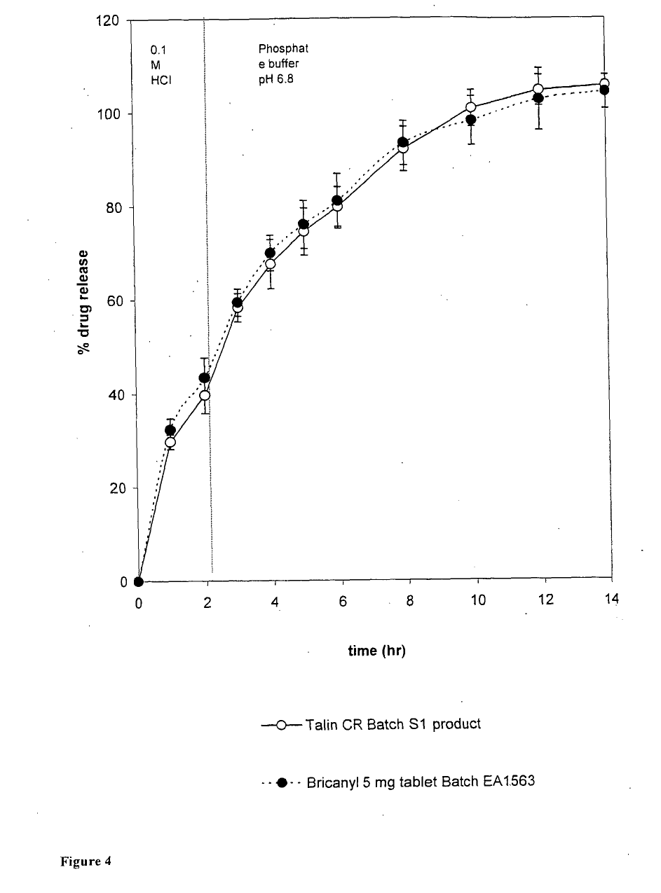

FIG. 4 shows the time course of Terbutaline sulfate release from controlled-release tablets (Batch S1, herein referred to as “Talin” or “Talin XR”) compared to Bricanyl 5 mg tablets (Batch EA1536)

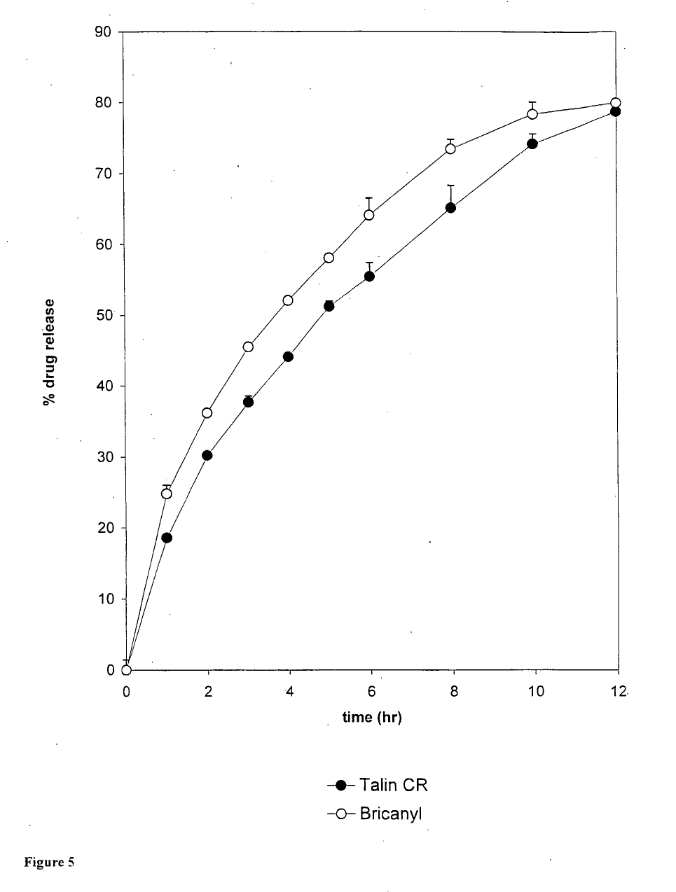

FIG. 5 shows the dissolution profile obtained from HPLC analysis of Terbutaline sulfate in deionized water compared to that of Bricanyl Durules 5 mg.

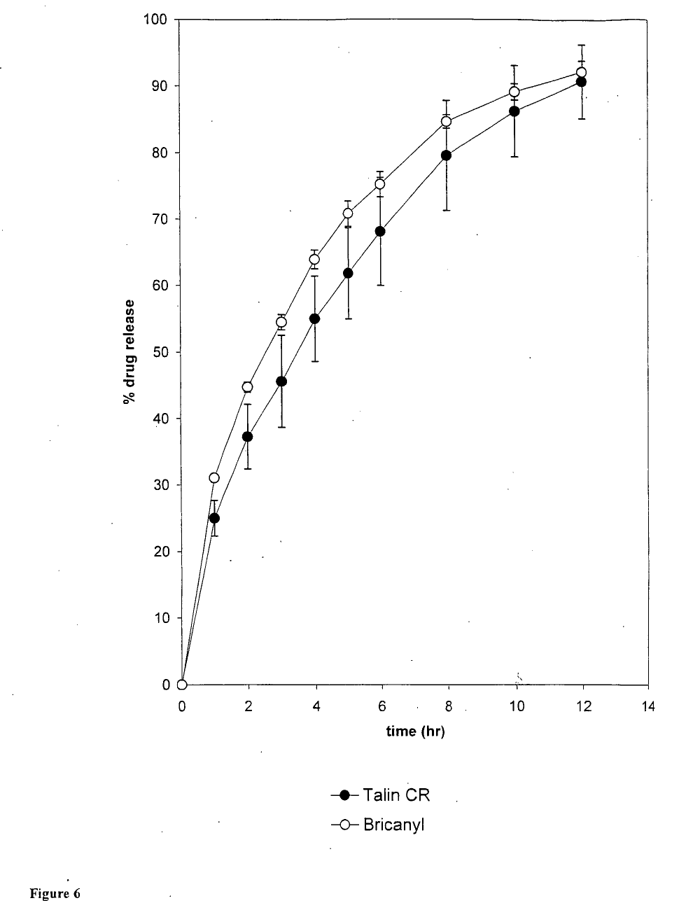

FIG. 6 shows the dissolution profile of Terbutaline sulfate obtained from HPLC analysis in 0.1 M HCl compared to that of Bricanyl Durules 5 mg.

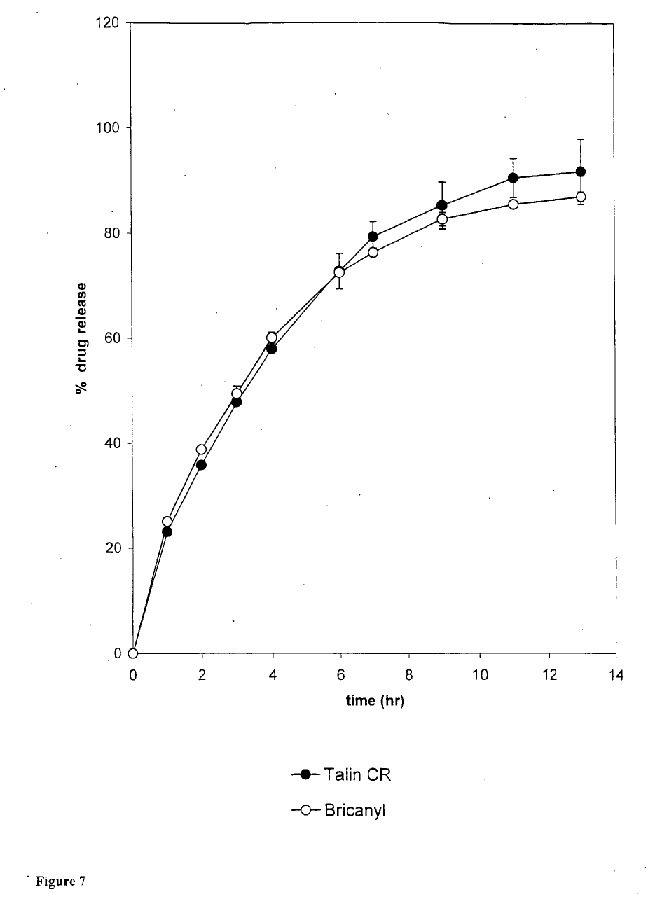

FIG. 7 shows the dissolution profile of Terbutaline sulfate obtained from HPLC analysis in phosphate buffer, pH 6.8 compared to that of Bricanyl Durules 5 mg.

FIG. 8 shows the dissolution profile of Terbutaline sulfate obtained from UV spectrophotometric analysis in deionized water compared to that of Bricanyl Durules 5 mg.

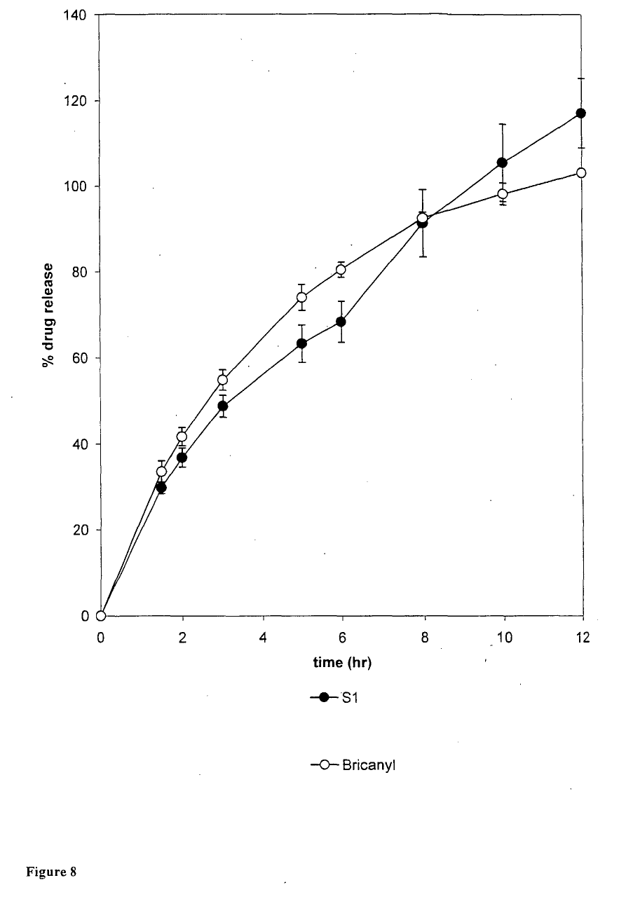

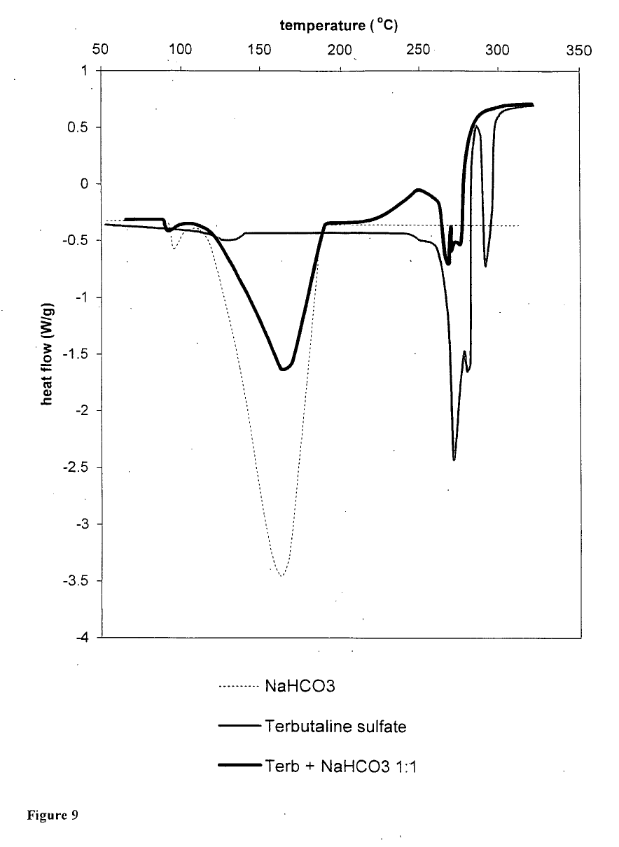

FIG. 9 shows DSC thermograms of sodium bicarbonate, Terbutaline sulfate and a physical mixture of Terbutaline sulfate and sodium bicarbonate 1:1 (w/w).

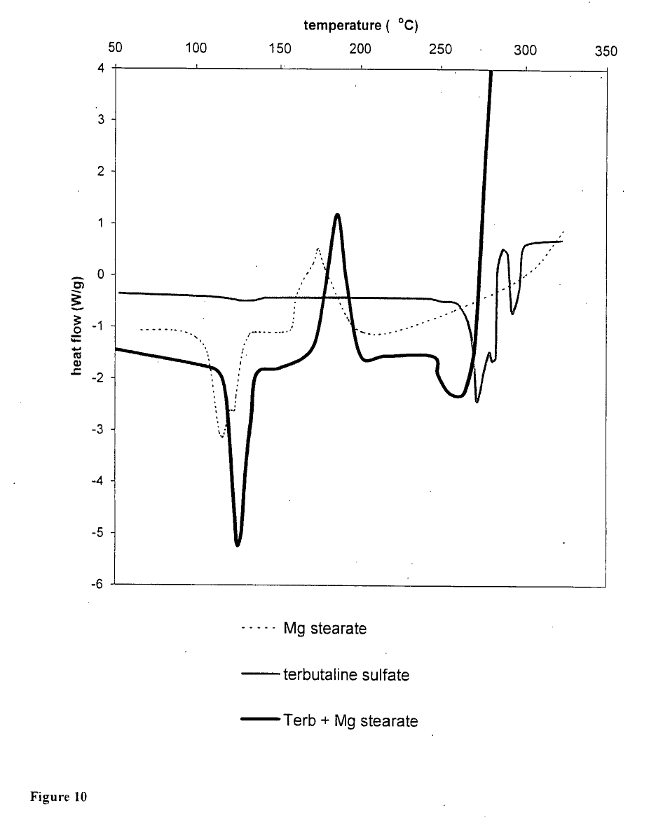

FIG. 10 shows DSC thermograms of magnesium stearate, Terbutaline sulfate and a physical mixture of Terbutaline sulfate and magnesium stearate 1:1 (w/w).

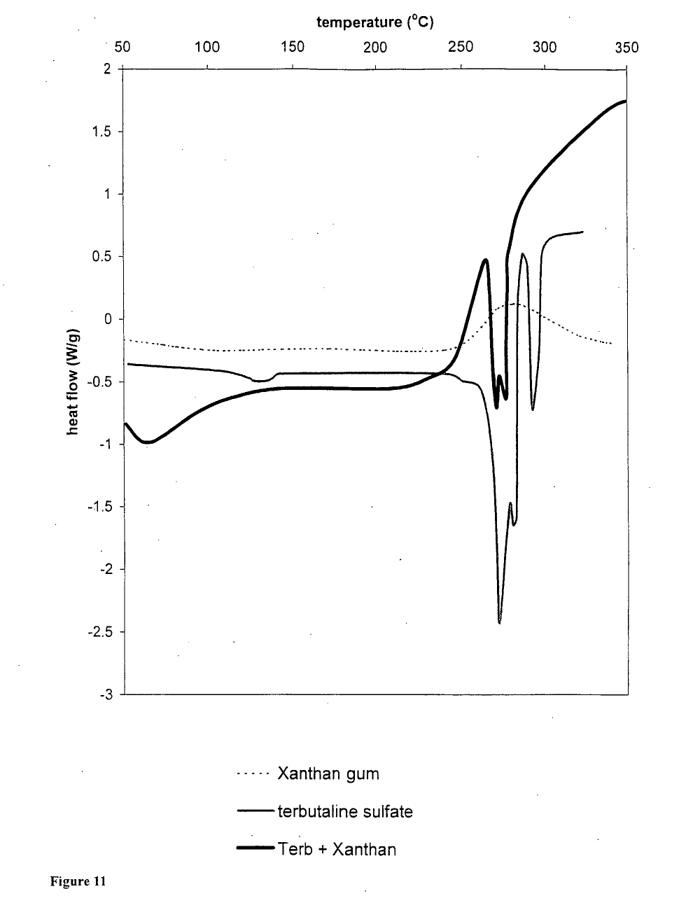

FIG. 11 shows DSC thermograms of xanthan gum, Terbutaline sulfate and a physical mixture of Terbutaline sulfate and xanthan gum 1:1 (w/w).

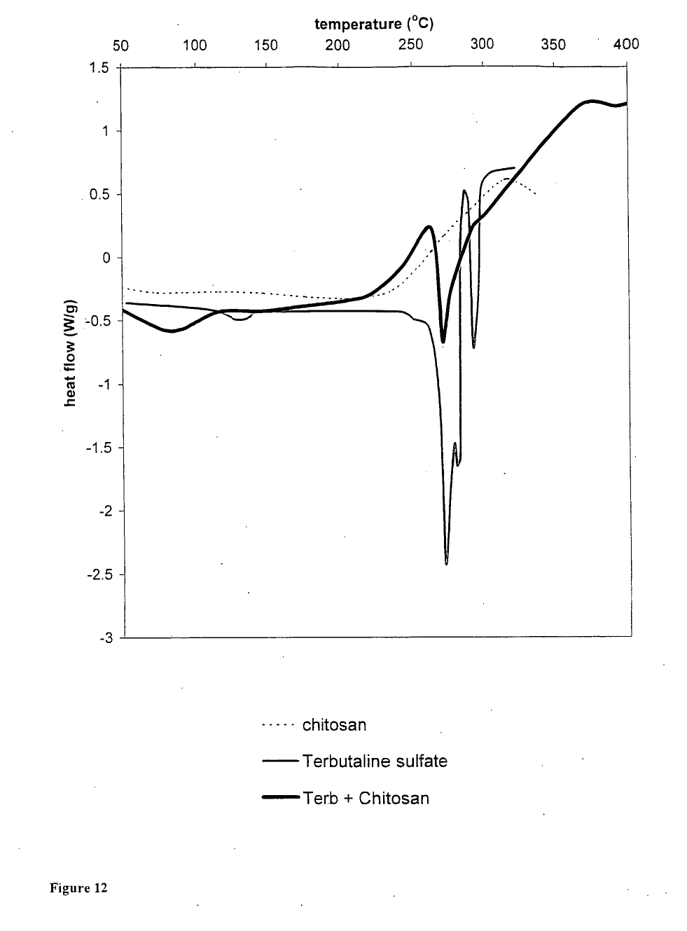

FIG. 12 shows DSC thermograms of chitosan, Terbutaline sulfate and a physical mixture of Terbutaline sulfate and chitosan 1:1 (w/w).

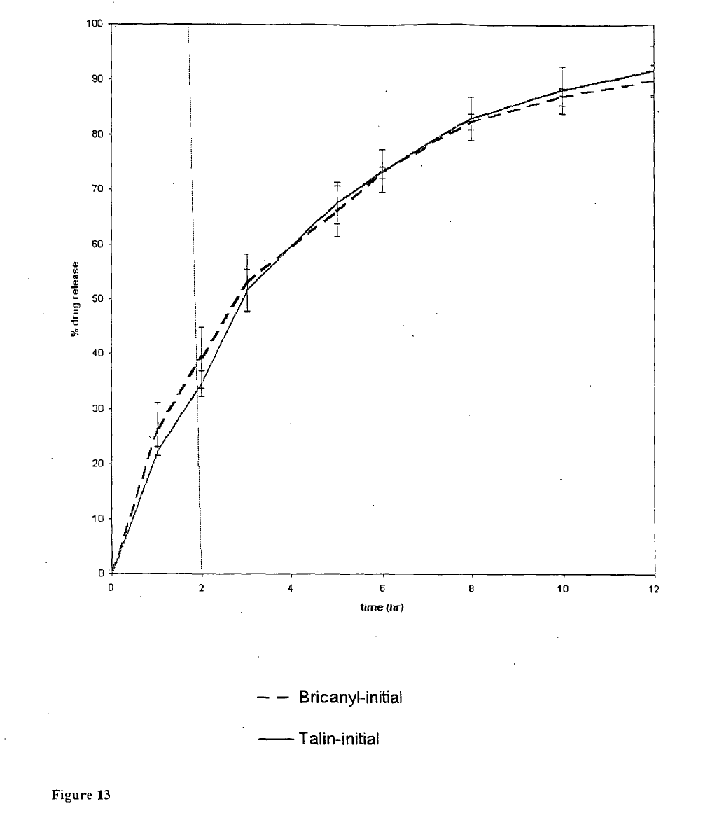

FIG. 13 shows the dissolution profile of Terbutaline sulfate from Bricanyl and Talin XR at room temperature.

FIG. 14 shows the dissolution profile of Terbutaline sulfate from Bricanyl and Talin XR stored under the following conditions: 40° C./75% RH, closed, for 3 months.

FIG. 15 shows a dissolution profile of Terbutaline sulfate from Bricanyl and Talin XR stored under the following conditions: 50° C., closed, for 3 months.

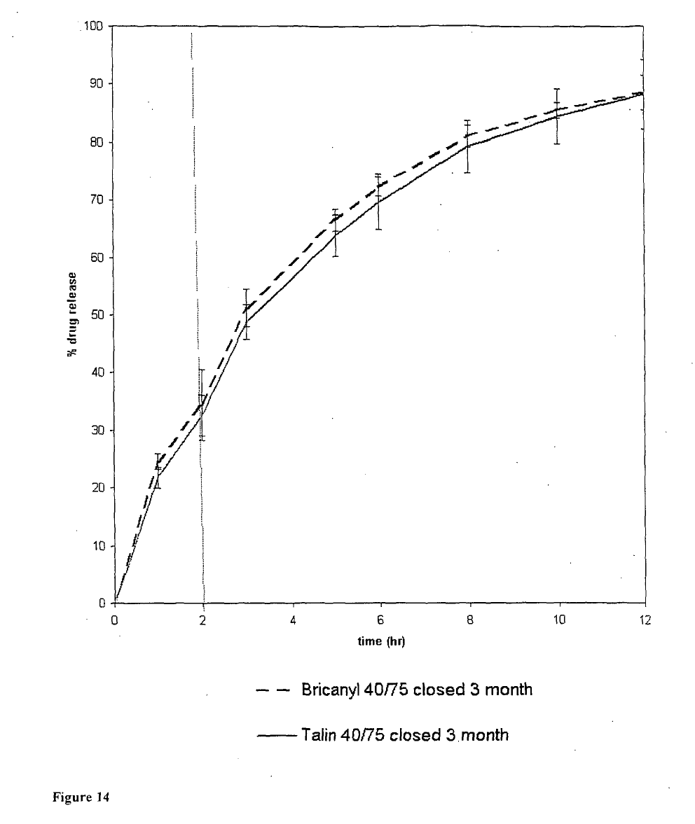

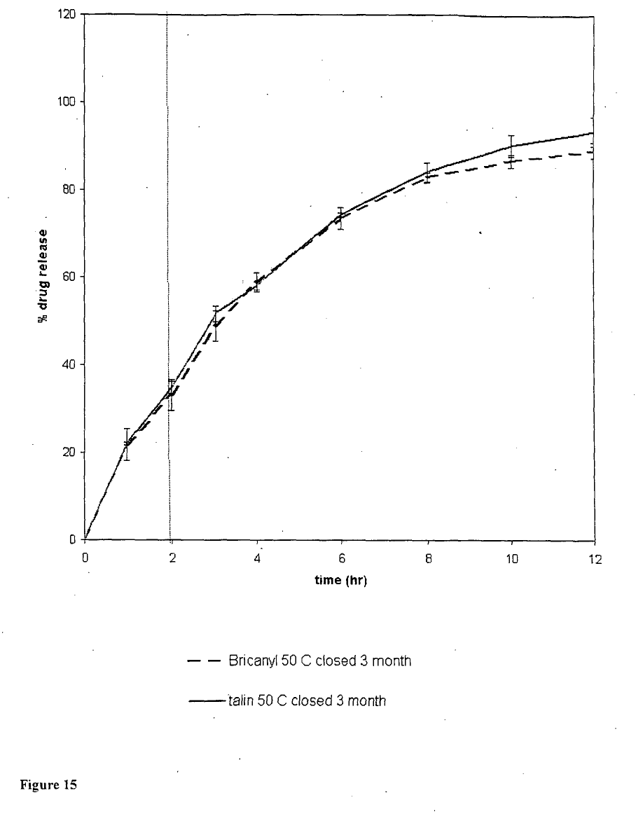

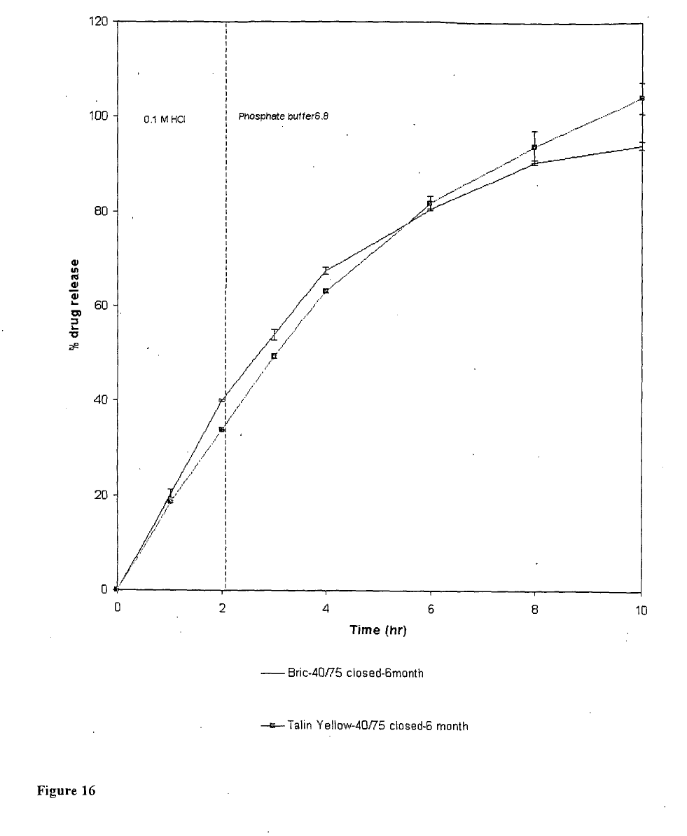

FIG. 16 shows a dissolution profile of Terbutaline sulfate from Bricanyl and Talin XR stored under the following conditions: 40° C./75% RH, closed, for 6 months.

FIG. 17 shows a dissolution profile of Terbutaline sulfate from Bricanyl and Talin XR under the following conditions: 40° C./75 RH, open, for 3 months.

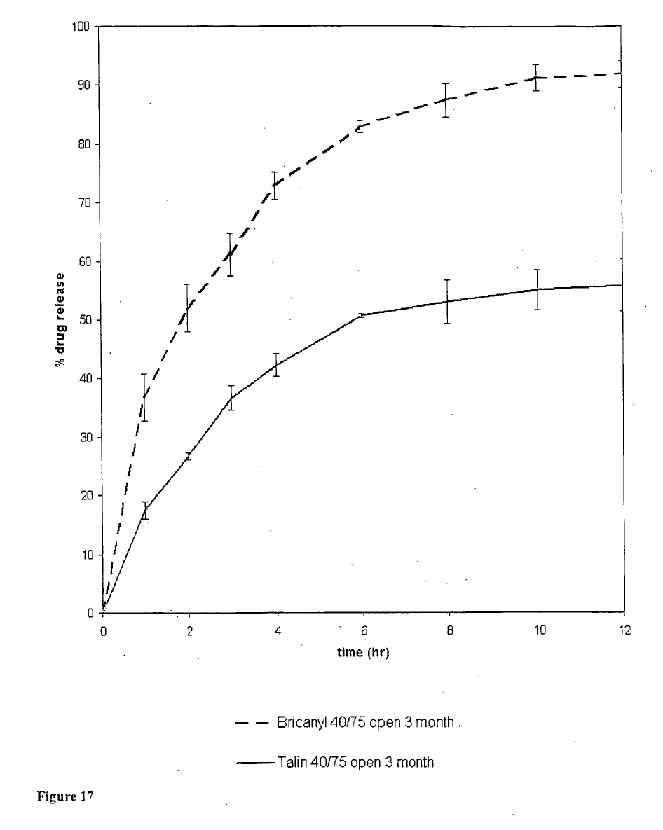

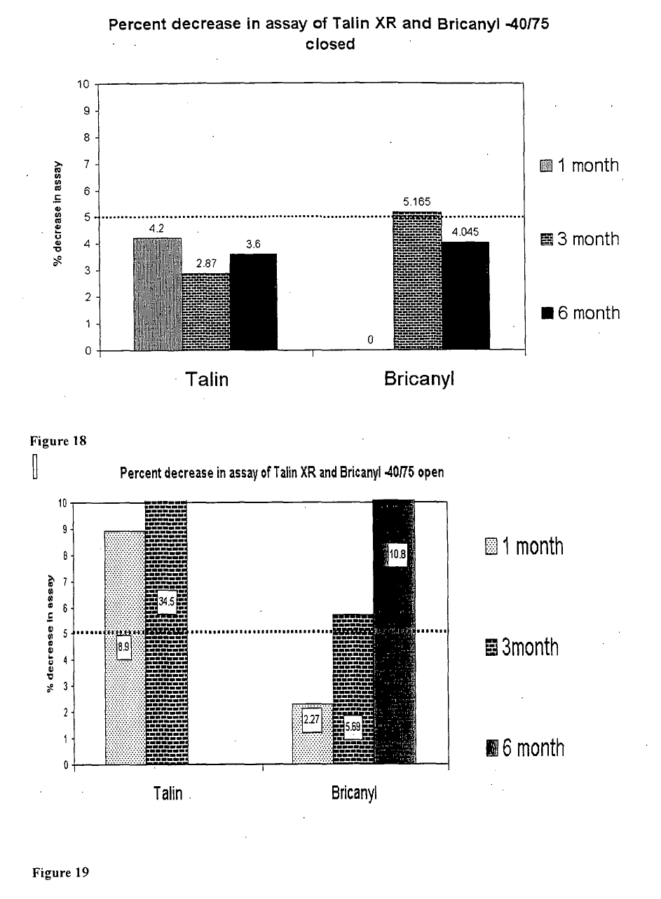

FIG. 18 shows the percent decrease in an assay of Talin XR and Bricanyl upon storage at 40° C./75% RH in closed bottles. Dashed line indicates the limits.

FIG. 19 shows the percent decrease in an assay of Talin XR and Bricanyl upon storage at 40° C./75% RH in open bottles. Dashed line indicates the limits.

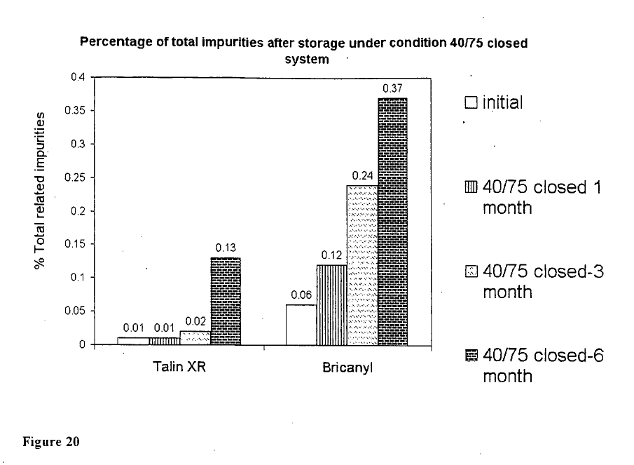

FIG. 20 shows percent total impurities of Talin XR and Bricanyl upon storage at 40° C./75% RH in closed bottles (B.P. limits <0.4%).

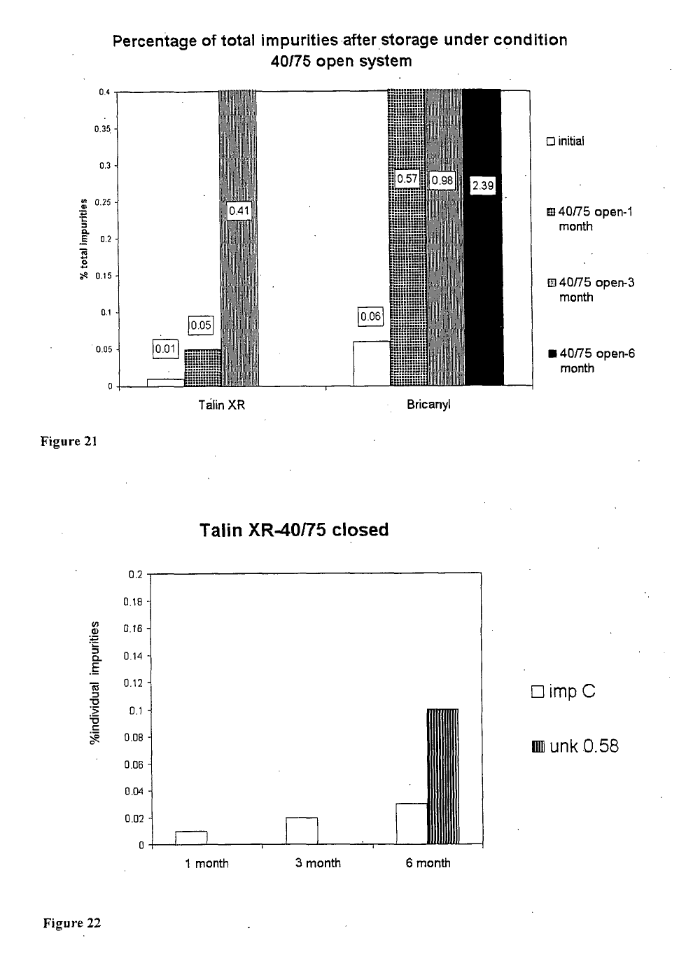

FIG. 21 shows percent total impurities of Talin XR and Bricanyl upon storage at 40° C./75% RH in open bottles (B.P. limits <0.4%).

FIG. 22 shows percent individual impurities of Talin XR upon storage at 40° C./75% RH in closed bottles (B.P. limits <0.2%).

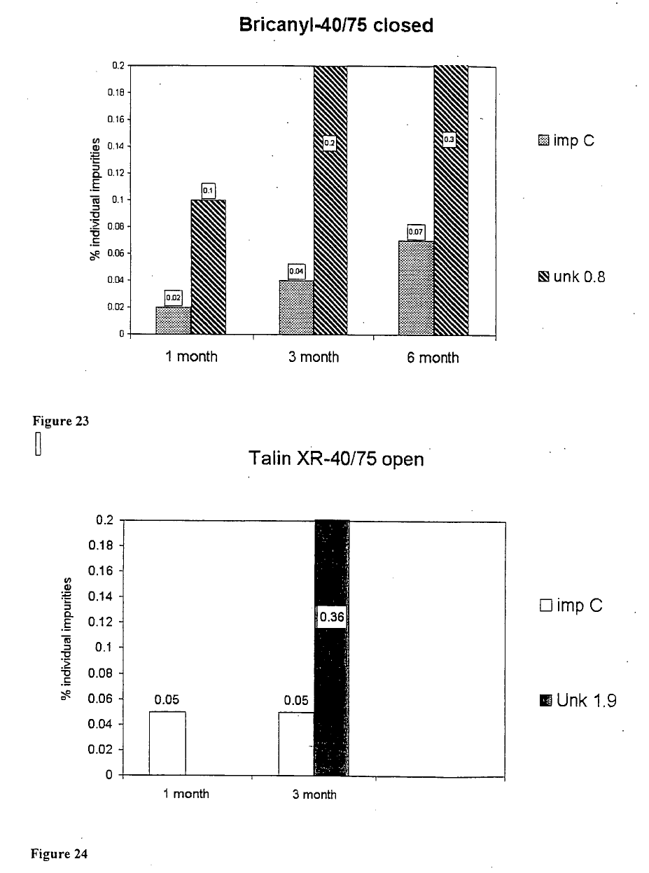

FIG. 23 shows percent individual impurities of Bricanyl upon storage at 40° C./75% RH in open bottles (B.P. limits <0.2%).

FIG. 24 shows percent individual impurities of Talin XR upon storage at 40° C./75% RH open bottles (B.P. limits <0.2%).

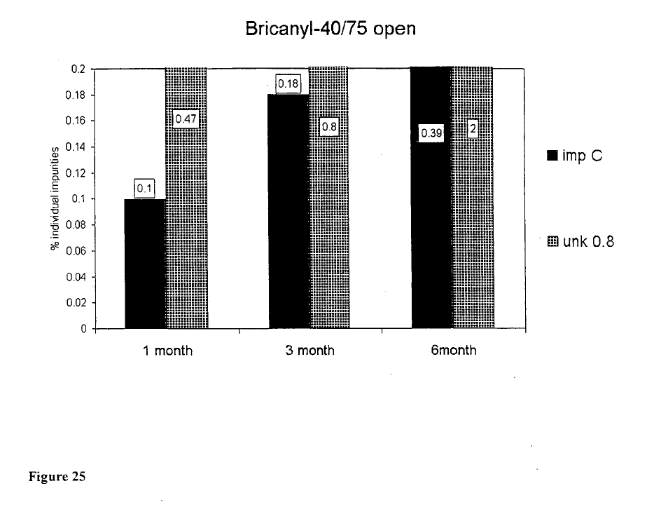

FIG. 25 shows percent individual impurities of Bricanyl upon storage at 40° C./75% RH in open bottles (B.P. limits <0.2%).

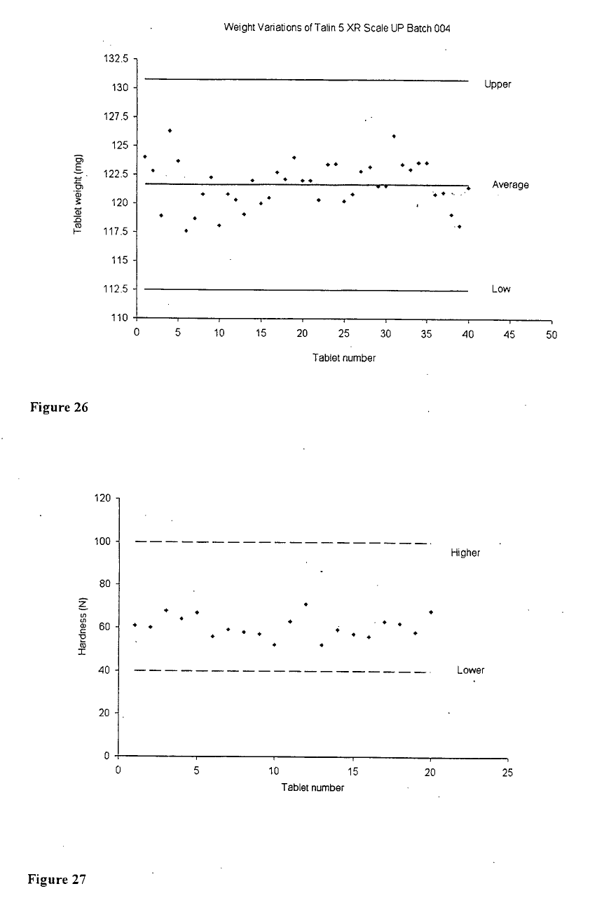

FIG. 26 shows a weight variation plot of 40 tablet representative samples of Talin XR BN 04.

FIG. 27 shows a tablet hardness (N) plot of 20 tablet representative samples of Talin XR BN 04.

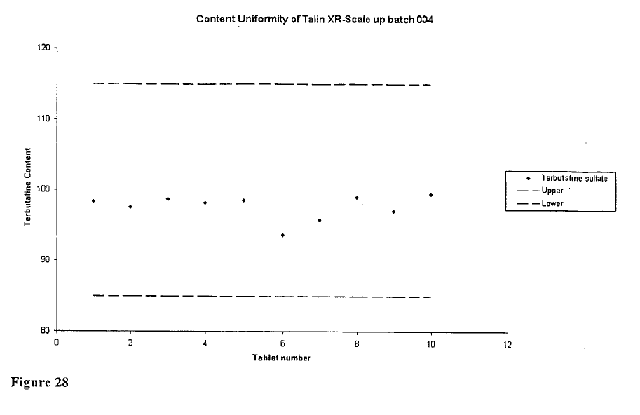

FIG. 28 shows a content uniformity plot of 10 tablet representative samples of Talin XR BN 04.

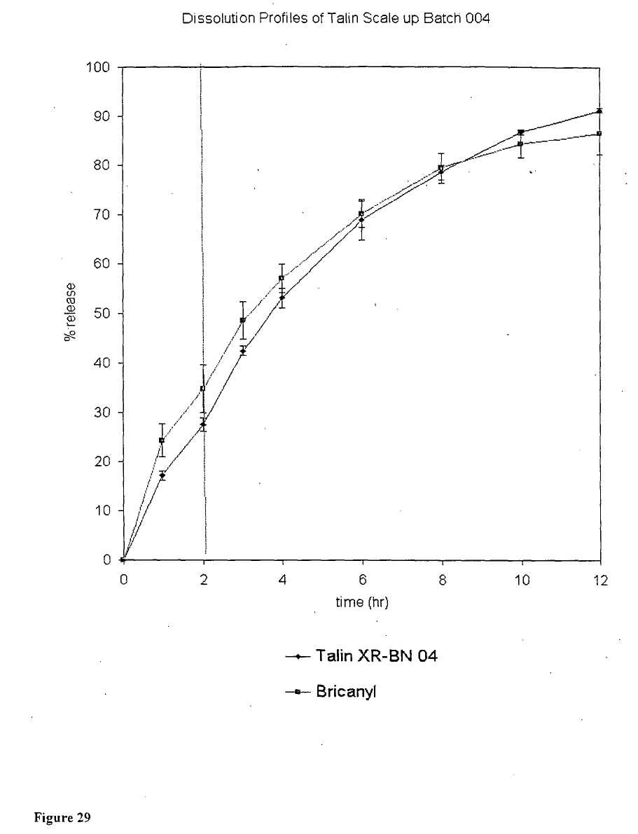

FIG. 29 shows a dissolution profile of Talin XR BN 04 compared to Bricanyl 5 mg tablets.

### EXAMPLE 1

### Investigation of Polymer Combination Controlled-Release Behavior

### Preparation of a Controlled-Release Tablet

The system contains 5 mg Terbutaline sulfate and 15 mg release modifying excipient (acidic: tartaric acid, or basic: sodium bicarbonate, or neutral: Avicel PH101) and 60 mg release retarding polymers per each single matrix. The polymer mixtures were CH:XG 1:1 (w/w). The drug/excipient to polymer mixture ratio was 1:3 (see Table 1). Tablets of 7 mm in diameter were prepared by a direct compression method. Components of each tablet were geometrically mixed by porcelain mortar and pestle for about 10 min before compression. Biplanar tablets were manufactured by compression of powder mixtures, applying a pressure of about 443 MPa for 15 sec by a hydraulic press.

### In Vitro Dissolution Test

The USP apparatus 1 (Basket) was used. The vessels were placed in a water bath regulated to maintain a temperature of 37±0.5° C. during the test. A fitted cover was used on the vessel to prevent any evaporation during the test. All tablets were subjected to 500 ml 0.1 M HCl USP solution for 2 hrs. The acidic medium was decanted and replaced with 500 ml phosphate buffer pH 6.8 USP solution for the rest of the dissolution time. The speed of dissolution test was set at 100 rpm. At specified time intervals, 6 ml aliquots were withdrawn. At each time interval, an aliquot equal in volume to the withdrawn sample was replaced to maintain the original volume of dissolution medium. The reported dissolution results were the average mean of three readings. Samples of were taken and analyzed using an UV spectrophotometer. Absorbance data were measured at 235 nm, 2^{nd} derivative. All calculations were performed with reference to the pre-pared calibration curves (see Table 2).

TABLE 1
Summary of different formulae used for the development of a
Terbutaline sultate controlled-release product compared
to a Bricanyl 5 mg tablet.

| | |
|---|---|
| Constituents | Formula no. |

| (mg/tablet) | 1 | 2 | 3 | 4* |
|---|---|---|---|---|
| Terbutaline sulfate | 5 | 5 | 5 | 5 |
| Avicel PH 200 | 15 | 0 | 0 | 0 |
| Sodium bicarbonate | 0 | 15 | 0 | 0 |
| Tartaric acid | 0 | 0 | 15 | Yes |
| Chitosan | 30 | 30 | 30 | 0 |
| Xanthan gum | 30 | 30 | 30 | 0 |
| Total | 80 | 80 | 80 | 143 |

*Reference commercial controlled-release product (Bricanyl Durules ® 5 mg).

A Commercially available “Bricanyl 5 mg tablet” and “Bricanyl Durules 5 mg”, respectively, (manufactured by AstraZeneca, Sweden; Lot no. EA1563, Man 2003-01, Exp 2006-01), was used as a reference. “Bricanyl®” and “Bricanyl Durules®” are registered trade marks of AstraZeneca. One tablet contains 5 mg Terbutaline sulfate and the following excipients: stearyl alcohol, ethyl cellulose, colloidal silicone dioxide, tartaric acid and polyvinyl chloride according to VIDAL.

TABLE 2
Summary of calibration curves of Terbutaline sulfate
in 0.1 M HCl and in phosphate buffer, pH 6.8.

| | λₘₐₓ (nm) | | | | |
|---|---|---|---|---|---|
| | 2^{nd} derivative | | | Conc. range | |
| Media | (non smooth) | Slope | Intercept | (mg/100 ml) | R2 |
| 0.1 M HCl | 235 | 0.0023 | 2E−5 | 0.0811-5.00 | 0.9999 |
| Phosphate | 235 | 0.0024 | 1E−6 | 0.04-2.50 | 0.9997 |
| buffer, | | | | | |
| pH 6.8 | | | | | |

Comparison of Drug Release with a Reference Commercial Product

The dissolution profile of a Bricanyl 5 mg tablet from AstraZeneca (Sweden) was used as a reference. The reference dissolution profile was compared with the samples via f2 factor of SUPAC (scale-up and postapproval change) suggested by FDA. Dissolution profiles of the reference samples would be considered similar when f2 is larger than 50:

$$F = 50 \cdot log [ \frac{100}{\sqrt{1 + \frac{{\sum }_{t = 1}^{t = n} {[ Rt - Tt ]}^{2}}{n}}} ]$$

The use of CH/XG mixtures resulted in a better controlling release than the previous combinations (see FIG. 1), especially when a basic excipient (sodium bicarbonate) was used in the controlled-release formula. Based on calculations of similarity factor (f2), none of these formulae resulted in a release similar to Bricanyl, as shown in Table 3. But f2 indicated that formula 2 might be the best among these. Thus, further development should be conducted on formula 2. The only thing now to modify is the D/E:P ratio, it should be increased until a release behavior similar to Bricanyl is obtained.

TABLE 3
Similarity factor of calculations of the 6 formulae.

| Formula no. | Similarity factor (f2) |
|---|---|
| 1 | 32.77 |
| 2 | 40.51 |
| 3 | 28.17 |
| 4 | 30.00 |
| 5 | 34.64 |
| 6 | 26.29 |

### EXAMPLE 2

### Screening Out the Suitable D/E:P Ratio of the Polymer Mixture (P) (Chitosan/Xanthan 1:1) and the Excipient (E) of Sodium Bicarbonate

The system contains 5 mg Terbutaline sulfate and 15 mg release modifying excipient (sodium bicarbonate) and release retarding polymer mixture. The polymer mixture was CH:XG in a ratio of 1:1. The drug/excipient to polymer mixture (D/E:P) ratio was 1:5, 1:6, and 1:8, as shown in Table 4. Tablets of 7 mm in diameter were prepared by a direct compression method. Components of each tablet were geometrically mixed by porcelain mortar and pestle for about 10 min before compression. Biplanar tablets were manufactured by compression of powder mixtures, applying a pressure of about 443 MPa for 15 sec by a hydraulic press. The in vitro dissolution conditions and the analysis methods were similar to that mentioned in Example 1.

TABLE 4
Summary of the formulae used for the development of a Terbutaline
sulfate controlled-release product compared to a Bricanyl 5 mg tablet.

| Constituents | Formula no. | | | | | |
|---|---|---|---|---|---|---|
| (mg/tablet) | 1 | 2 | 3 | 4 | 5 | 6* |
| Terbutaline sulfate | 5 | 5 | 5 | 5 | 5 | 5 |
| Chitosan | 15 | 15 | 15 | 0 | 0 | |
| Xanthan gum | 50 | 60 | 80 | 37.5 | 75 | |
| Sodium bicarbonate | 50 | 60 | 80 | 37.5 | 75 | |
| Total | 120 | 140 | 180 | 80 | 155 | 144 |
| D:P ratio | | | | 1:15 | 1:30 | |
| D/E:P ratio | 1:5 | 1:6 | 1:8 | | | |

*Bricanyl Durules ® 5 mg

The release from CH/XG matrix system was similar to that of Bricanyl in formulae 1, 2 and 3. Thus, increasing the D/E:P from 1:3 to 1:5 resulted in a release pattern much similar to that of Bricanyl. This is also demonstrated by the estimation of similarity factor f2, where 12 was higher than 50 in these formulae (see Table 5). Drug release from the Bricanyl depends on diffusion mechanism. Since the tablet stays intact with no increase in its dimension allover the process of dissolution, the surface area is constant during the process of dissolution. On the other hand, Talin XR tablet (for a definition, see Example 3 below) swells and the surface area is increasing with time in an acidic environment. In phosphate buffer, the surface area becomes constant and from which drug diffuses through. Also, a floating system is developed in the acidic medium due to the presence of sodium bicarbonate. This is beneficial since it will increase the tablet resident time in the gut.

To illustrate the advantage of the use of sodium bicarbonate as a filler in CH/XG mixture, formulae 4 and 5 were investigated with high D:P ratios of 1:15 and 1:30, respectively. Sodium bicarbonate was not incorporated in these formulae. The drug release retardation power of these formulae was decreased tremendously although a very high concentration of the retarding polymer mixture was used (see FIG. 3). The similarity factor 12 of formulae 4 and 5 was lower than 50, as shown in Table 5. This indicates that the release behavior was not similar to Bricanyl. Ultimately, the use of small concentrations of sodium bicarbonate increases the retardation power of CH/XG system and resulted in a system of similar drug release to Bricanyl.

Consequently, the cost effective formula that resulted in a release behavior similar to Bricanyl would be D/E:P 1:5. This formula must be enlarged for full investigation in small-scale experiment using a single punch machine. Then, if proven its quality, a scaling up program should be conducted.

TABLE 5
Similarity factor calculations of the 5 formulae.

| Formula no. | Similarity factor (f2) |
|---|---|
| 1 | 66.48 |
| 2 | 68.89 |
| 3 | 60.63 |
| 4 | 35.08 |
| 5 | 47.24 |

### EXAMPLE 3

### Conduction of a Small Scale Experiment Through Direct Compression of the Powder Mixture D/E:P Ratio 1:5 Using a Single Punch Machine

The system contains 5 mg Terbutaline sulfate and 15 mg release modifying excipient (sodium bicarbonate) and release retarding polymer mixture per each tablet. The polymer mixture was CH:XG in a ratio of 1:1. The drug/excipient to polymer mixture (D/E:P) ratio was 1:5 (see Table 6). Components of each tablet were geometrically mixed for about 10 min before compression. Biplanar tablets of 7 mm in diameter were prepared by direct compression using a single punch machine. The batch was 500 g in size, and the batch number was referred to as S1. The proposed generic names for Batch S1 tablets is “Talin” and “Talin extended-release tablet” and “Talin XR”. Five tablets were selected randomly from Bricanyl Durules and Talin XR for physical characterizations (see tables 7 and 8, respectively). The in vitro dissolution conditions and the analysis methods were similar to those mentioned in Example 1.

TABLE 6
Summary of the formula (S1) used for the development of a
Terbutaline sulfate controlled-release product (Talin XR)
compared to a Bricanyl 5 mg tablet.

| Formula | |
|---|---|
| Constituents (mg/tablet) | |
| Terbutaline sulfate | 5 |
| Sodium bicarbonate | 15 |
| Xanthan gum | 49.5 |
| Chitosan | 49.5 |
| Magnesium Stearate | 1 |
| Total | 120 |
| D/E:P ratio | 1:5 |

TABLE 7
Physical properties of a Bricanyl 5 mg tablet (Batch EA1563).

| | Tablet no. | | | | | | Standard |
|---|---|---|---|---|---|---|---|
| | 1 | 2 | 3 | 4 | 5 | Average | deviation |
| Diameter (mm) | 7 | 7 | 7 | 7 | 7 | 7 | 0 |
| Thickness (mm) | 2.7 | 2.7 | 2.7 | 2.7 | 2.7 | 2.7 | 0 |
| Weight (mg) | 144.30 | 141.30 | 143.70 | 143.70 | 143.00 | 143.20 | 1.158 |
| Hardness (N) | 71.00 | 63.00 | 62.00 | 65.00 | 67.00 | 65.60 | 3.578 |

TABLE 8
Physical properties of a Talin controlled-release tablet (Batch S1).

| | Tablet no. | | | | | | Standard |
|---|---|---|---|---|---|---|---|
| | 1 | 2 | 3 | 4 | 5 | Average | deviation |
| Diameter (mm) | 7 | | 7 | 7 | 7 | 7 | 0 |
| Thickness (mm) | 2.39 | 2.37 | 2.25 | 2.35 | 2.31 | 2.334 | 0.55 |
| Weight (mg) | 118.80 | 115.00 | 118.90 | 120.20 | 120.30 | 118.64 | 2.152 |
| Hardness (N) | 85.00 | 80.00 | 81.00 | 87.00 | 80.00 | 82.60 | 3.209 |

The release from Talin XR release matrix system (Batch S1) was similar to that of Bricanyl, (see FIG. 4). Thus, the D/E:P 1:5 resulted in a release pattern much similar to that of Bricanyl Durules. This is also demonstrated by the estimation of similarity factor f2, where f2 was higher than 50 (around 80) in this formula (see Table 9).

The use of gradient dissolution media of two stages is an important criterion to judge Terbutaline sulfate release. The use of gradient in vitro dissolution media was found to be more correlated with the in vivo data than a single dissolution medium. The in vivo/in vitro correlation was established between the time for 80% of the drug release in gradient in vitro dissolution media and some in vivo parameters such as AUC and Cₘₐₓ (D. Torres, G. Garcia-Encina, B. Seijo, and J. Vila-Jato, Biopharmaceutical Evaluation of Microencapsulated Ion-Exchange Resins Containing Diclofenac. Eur. J. Pharm. Biopharm. 41, 127-131, 1995; C. Liu, Y. Kao, S. Chen, T. Sokoloski, and M. Sheu, In-vitro Studies of Diclofenac Sodium Controlled-Release Matrix Tablets. J. Pharm. Pharmacol. 47, 360-364, 1995; A. Abu-Mahadi, In-vitro and In-vivo Evaluation of Modified Release Diclofenac Sodium Dosage Form (M.Sc. thesis). Supervised by Dr. Naji Najib and Dr. Khouloud Alkhamis. Submitted to the Jordan University of Science and Technology, Irbid, Jordan, in December 1999).

Ultimately, similarity between Talin XR and Bricanyl Durules using gradient pH dissolution was confirmed. However, similarity should be maintained if a single dissolution medium was applied, for example, in 0.1 M HCl, phosphate buffer, pH 6.8 and water. This also could be an important issue when postapproval changes are needed as pointed out in the Guidance of Industry of SUPAC-MR changes in the formulation.

TABLE 9
Determination of similarity factor f2 according to FDA regulations.

| Time | Response | Response | | |
|---|---|---|---|---|
| (hrs) | (Reference product) | (Test product) | Difference | Difference 2 |
| 1 | 32.36 | 29.72 | 2.64 | 6.96 |
| 2 | 43.47 | 39.72 | 3.75 | 14.06 |
| 3 | 59.44 | 58.33 | 1.11 | 1.23 |
| 4 | 70.00 | 67.64 | 2.36 | 5.57 |
| 5 | 76.11 | 74.58 | 1.53 | 2.33 |
| 6 | 81.11 | 79.86 | 1.25 | 1.56 |
| 8 | 93.33 | 92.08 | 1.25 | 1.56 |
| 10 | 98.06 | 100.70 | −2.64 | 6.96 |
| 12 | 102.64 | 104.58 | −1.94 | 3.78 |
| 14 | 104.17 | 105.56 | −1.39 | 1.93 |

| | |
|---|---|
| Sum (Difference 2) = | 45.97 |
| ((Sum(Difference2)/n) + 1) 0.5 | 2.37 |
| 100/(Sum(Difference\hat{ }2)/n + 1) 0.5 | 42.27 |
| F = 50 * Log[100/Sum(Difference2)/n + 1) 0.5)] = | 81.30 |

### EXAMPLE 4

### Similarity in Release Between Talin XR and Bricanyl Durules Using Different Single Dissolution Media of Water

The in vitro dissolution conditions were similar to those mentioned in Example 1. However, a single dissolution medium of water, 0.1 M HCl and phosphate buffer, pH 6.8 was used.

The details of the analysis conditions are shown in Table 10. The method of analysis used is validated internally. All calculations of HPLC analysis were performed for the average of two separate experiments. Standard samples were prepared according to the theoretical concentration, assuming full release of the active drug from the matrix into the dissolution medium.

TABLE 10
HPLC analysis conditions of dissolution sample.

| Active material | Terbutaline sulfate |
|---|---|
| Mobile phase (M.P.) | a mixture of 0.15 M ammonium acetate |
| | and glacial acetic acid (pH 4, 96:4, v/v); |
| | the mobile phase was filtered and |
| | degassed by sonication before use; |
| Flow rate (ml/min) | 2 ml/min |
| Loop volume (μl) | 100 μl |
| Column | Hypersil 100 ODS, C18, 5 μm, |
| | 10 cm * 4.6 mm ID (Shandon, England) |
| Wave length (nm) | 270 nm |
| Final conc. standard (mg %) | 1.0 mg % |

### Assay of Bricanyl and Talin XR

Twenty tablets were crushed using mortar and pestle. Accurately weighted quantities of powdered tablets equivalent to 15 mg Terbutaline sulfate were shaken with 100 ml water for 15 min and centrifuged. Two ml were taken and volume was completed to 25 ml with phosphate buffer, pH 6.8. The final concentration of the sample is around 1.2 mg/100 ml. The sample was analyzed using the HPLC method according to Table 10.

A standard was prepared of Terbutaline sulfate (75 mg) in 100 ml water. Five ml were taken and volume was completed to 25 ml with water. Four ml were taken and the volume was completed with phosphate buffer, pH 6.8. The final concentration of the standard is 1.2 mg/100 ml. The standard was analyzed using the HPLC method according to Table 10. Two repetitions were made for each sample and standard, and the mean was taken for statistical analysis.

In case of water, the hydrophilic polymers will dissolve with time until the tablet (Talin XR) has completely disappeared. This did not happen in the hydrophobic matrix of Bricanyl. It was intact all the time irrespective of the pH of the dissolution medium. Nevertheless, the similarity factor f2 showed that these two formulations showed similar release pattern (f2>50).

The dissolution profile analyzed on the basis of the HPLC method showed about 80% release after 12 hrs of dissolution (see FIG. 5). The estimated f2 was higher than 50, as shown in Table 12. This indicates the similarity in release. Talin XR was similar in release to Bricanyl if water was used as a dissolution medium.

TABLE 11
Assay of Terbutaline sulfate in Talin XR and Bricanyl Durules initial condition.

| | | Weight of | Average | Sample | | | % Assay |
|---|---|---|---|---|---|---|---|
| | | 5 units | weight unit | weight | Final conc. | Average | Mean |
| Sample | Condition | (mg) | (mg) | (mg) | (mg/100 ml) | response | (CV %) |
| Talin CR | initial | 603 | 120.6 | 180.2, | 1.201, | 160223, | 97.4 |
| | | | | 180 | 1.201 | 157673 | (1.1) |
| Bricanyl | initial | 722 | 144.2 | 217, | 1.202, | 165992, | 101.3 |
| | | | | 217 | 1.202 | 166573 | (0.3) |

TABLE 12
Determination of similarity factor f2 according to FDA regulations
obtained according to HPLC analysis.

| Time | Response | Response | | |
|---|---|---|---|---|
| (hrs) | (Reference product) | (Test product) | Difference | Difference 2 |
| 1 | 24.80 | 18.60 | 6.20 | 38.44 |
| 2 | 36.23 | 30.22 | 6.01 | 36.08 |
| 3 | 45.52 | 37.72 | 7.80 | 60.85 |
| 4 | 52.05 | 44.13 | 7.93 | 62.81 |
| 5 | 58.10 | 51.22 | 6.88 | 47.31 |
| 6 | 64.10 | 55.45 | 8.65 | 74.82 |
| 8 | 73.40 | 65.10 | 8.30 | 68.89 |
| 10 | 78.36 | 74.15 | 4.21 | 17.68 |
| 12 | 79.90 | 78.70 | 1.20 | 1.44 |

| | |
|---|---|
| Sum (Difference 2) = | 408.32 |
| ((Sum(Difference2)/n) + 1)0.5 | 6.81 |
| 100/(Sum(Difference\hat{ }2)/n + 1)0.5 | 14.69 |
| F = 50 * Log[100/(Sum(Difference2)/n + 1) 0.5)] = | 58.34 |

### EXAMPLE 5

The objective of this example is to examine the similarity in release between Talin XR and Bricanyl Durules in three different single dissolution media (0.1 M HCl, water, phosphate buffer, pH 6.8).

The dissolution conditions and method of analysis are similar to that mentioned in Example 1 and Example 4, respectively.

The dissolution profile in 0.1 M HCl medium of Talin and Bricanyl are shown in FIG. 6. The similarity factor was >50, as shown in Table 13. Thus, Talin XR is similar in release to Bricanyl if 0.1 M HCl was used as a dissolution medium.

FIG. 7 shows the dissolution profile of Talin XR and Bricanyl in phosphate buffer, pH 6.8. The dissolution profiles were similar, as indicated by the similarity factor (f2>50; see Table 14). FIG. 8 shows the dissolution profile of Talin XR and Bricanyl in water. The dissolution profiles were similar, as indicated by the similarity factor (f2>50; see Table 15).

TABLE 13
Determination of similarity factor f2 according to FDA regulations
obtained according to HPLC analysis.

| Time | Response | Response | | |
|---|---|---|---|---|
| (hrs) | (Reference product) | (Test product) | Difference | Difference 2 |
| 1 | 31.05 | 25.01 | 6.04 | 36.42 |
| 2 | 44.76 | 37.26 | 7.50 | 56.18 |
| 3 | 54.50 | 45.60 | 8.90 | 79.21 |
| 4 | 63.92 | 55.01 | 8.91 | 79.36 |
| 5 | 70.85 | 61.85 | 9.00 | 81.00 |
| 6 | 75.25 | 68.15 | 7.10 | 50.41 |
| 8 | 84.69 | 79.55 | 5.14 | 26.42 |
| 10 | 89.15 | 86.25 | 2.90 | 8.41 |
| 12 | 92.20 | 90.70 | 1.50 | 2.25 |

| | |
|---|---|
| Sum (Difference 2) | 419.66 |
| ((Sum(Difference2)/n) + 1)0.5 | 6.90 |
| 100/(Sum(Difference\hat{ }2)/n + 1)0.5 | 14.49 |
| F = 50 * Log[100/(Sum(Difference2)/n + 1)0.5)] = | 58.05 |

TABLE 14
Determination of similarity factor f2 according to FDA regulations
obtained according to HPLC analysis.

| Time | Response | Response | | |
|---|---|---|---|---|
| (hrs) | (Reference product) | (Test product) | Difference | Difference 2 |
| 1 | 25.0625 | 23.0995 | 1.96 | 3.85 |
| 2 | 38.8195 | 35.815 | 3.00 | 9.03 |
| 3 | 49.4785 | 47.8625 | 1.62 | 2.61 |
| 4 | 60.0855 | 57.939 | 2.15 | 4.61 |
| 6 | 72.4345 | 72.715 | −0.28 | 0.08 |
| 7 | 76.29 | 79.302 | −3.01 | 9.07 |
| 9 | 82.6615 | 85.2795 | −2.62 | 6.85 |
| 11 | 85.501 | 90.527 | −5.03 | 25.26 |
| 13 | 86.9055 | 91.6545 | −4.75 | 22.55 |

| | |
|---|---|
| Sum (Difference 2) = | 61.36 |
| ((Sum(Difference2)/n) + 1) 0.5 | 2.80 |
| 100/(Sum(Difference\hat{ }2)/n + 1) 0.5 | 35.76 |
| F = 50 * Log[100/(Sum(Difference2)/n + 1) 0.5)] = | 77.67 |

TABLE 15
Determination of similarity factor f2 according to FDA regulations
obtained according to HPLC analysis.

| Time | Response | Response | | |
|---|---|---|---|---|
| (hrs) | (Reference product) | (Test product) | Difference | Difference 2 |
| 1 | 24.80 | 18.60 | 6.20 | 38.44 |
| 2 | 36.23 | 30.22 | 6.01 | 36.08 |
| 3 | 45.52 | 37.72 | 7.80 | 60.85 |
| 4 | 52.05 | 44.13 | 7.93 | 62.81 |
| 5 | 58.10 | 51.22 | 6.88 | 47.31 |
| 6 | 64.10 | 55.45 | 8.65 | 74.82 |
| 8 | 73.40 | 65.10 | 8.30 | 68.89 |
| 10 | 78.36 | 74.15 | 4.21 | 17.68 |
| 12 | 79.90 | 78.70 | 1.20 | 1.44 |

| | |
|---|---|
| Sum (Difference 2) = | 408.32 |
| ((Sum(Difference2)/n) + 1) 0.5 | 6.81 |
| 100/(Sum(Difference\hat{ }2)/n + 1) 0.5 | 14.69 |
| F = 50 * Log[100/(Sum(Difference2)/n + 1)0.5)] = | 58.34 |

### EXAMPLE 6

The objective of this study is to check excipient compatibility of the optimal formula using a DSC instrument.

Samples investigated by DSC are summarized in Table 16. Samples of 5-10 mg in weight were placed in hermetically sealed DSC pans. Samples were scanned at a rate of 10° C./min, and thermograms were obtained. The thermal analyzer (DSC) was calibrated prior to use by indium. The calibration procedure was repeated many times prior to DSC runs.

TABLE 16
Summary of DSC runs applied for excipient compatibility study.

| | Substance | | | | |
|---|---|---|---|---|---|
| | Terbutaline | Xanthan | | Magnesium | |
| | sulfate | gum | | stearate | NaHCO₃ |
| DSC run | % w/w | % w/w | Chitosan | % w/w | % w/w |
| 1 | 100% | | | | |
| 2 | | 100% | | | |
| 3 | | | 100% | | |
| 4 | | | | 100% | |
| 5 | | | | | 100% |
| 6 | 50% | 50% | | | |
| 7 | 50% | | 50% | | |
| 8 | 50% | | | 50% | |
| 9 | 50% | | | | 50% |

FIGS. 9-12 summarizes the DSC thermograms obtained from each excipient (release modifying or non-release modifying) and a mixture of each excipient with Terbutaline sulfate in a ratio of 1:1 (w/w). The resulted thermograms of the mixtures showed no odd behavior during the heat scan. These scans were almost equivalent to additive thermograms resulted between the drug and each excipient. This may indicate excipient compatibility with Terbutaline sulfate. Thus, it is concluded that excipients are compatible with Terbutaline sulfate according to DSC results.

### EXAMPLE 7

The objective is an accelerated stability study of Talin XR compared to Bricanyl Durules under the storage conditions of 40° C./75% RH open, 40° C./75% RH closed, 50° C. in terms of assay, related and dissolution profiles.

Talin XR and Bricanyl have shown similar in vitro dissolution release pattern under closed conditions, as shown in FIGS. 13-16. Similarity factors (f2) for each storage condition were higher than 50 for samples stored under closed conditions, Tables 17-20. This indicates the similarity in release behavior between Talin XR and Bricanyl 5 mg tablet under closed conditions. On the other hand, humid conditions showed large variations in release behavior (see FIG. 17). The release pattern was dissimilar, since f2 was <50 (see Table 21). Thus, Bricanyl and Talin XR tablets should be stored protected from humidity.

TABLE 17
Determination of similarity factor f2 according to FDA regulations
at initial condition.

| Time | Response | Response | | |
|---|---|---|---|---|
| (hrs) | (Reference product) | (Test product) | Difference | Difference 2 |
| 1 | 26.465 | 22.385 | 4.08 | 16.65 |
| 2 | 39.355 | 34.585 | 4.77 | 22.75 |
| 3 | 52.975 | 51.67 | 1.31 | 1.70 |
| 5 | 66.04 | 67.555 | −1.52 | 2.30 |
| 6 | 73.115 | 73.415 | −0.30 | 0.09 |
| 8 | 82.35 | 82.97 | −0.62 | 0.38 |
| 10 | 87.02 | 88.23 | −1.21 | 1.46 |
| 12 | 89.865 | 91.75 | −1.89 | 3.55 |

| | |
|---|---|
| Sum (Difference 2) = | 48.89 |
| ((Sum(Difference2)/n) + 1) 0.5 | 2.67 |
| 100/(Sum(Difference\hat{ }2)/n + 1) 0.5 | 37.50 |
| F = 50 * Log[100/(Sum(Difference2)/n + 1) 0.5)] = | 78.70 |

TABLE 18
Determination of similarity factor f2 according to FDA regulations
at storage condition of 40° C./75% RH, closed, for 3 months.

| Time | Response | Response | | |
|---|---|---|---|---|
| (hrs) | (Reference product) | (Test product) | Difference | Difference 2 |
| 1 | 24.515 | 21.72 | 2.80 | 7.81 |
| 2 | 34.385 | 32.528 | 1.86 | 3.45 |
| 3 | 51.14 | 48.733 | 2.41 | 5.79 |
| 5 | 66.41 | 63.723 | 2.69 | 7.22 |
| 6 | 72.27 | 69.623 | 2.65 | 7.01 |
| 8 | 80.925 | 79.133 | 1.79 | 3.21 |
| 10 | 85.43 | 84.378 | 1.05 | 1.11 |
| 12 | 88.475 | 88.138 | 0.34 | 0.11 |

| | |
|---|---|
| Sum (Difference 2) = | 35.71 |
| ((Sum(Difference2)/n) + 1) 0.5 | 2.34 |
| 100/(Sum(Difference\hat{ }2)/n + 1) 0.5 | 42.78 |
| F = 50 * Log[100/(Sum(Difference2)/n + 1)0.5)] = | 81.56 |

TABLE 19
Determination of similarity factor f2 according to FDA regulations
at storage condition of 50° C., closed, for 3 months.

| Time | Response | Response | | |
|---|---|---|---|---|
| (hrs) | (Reference product) | (Test product) | Difference | Difference 2 |
| 1 | 21.7385 | 21.984 | −0.25 | 0.06 |
| 2 | 33.074 | 34.8905 | −1.82 | 3.30 |
| 3 | 48.8545 | 51.57 | −2.72 | 7.37 |
| 4 | 58.813 | 58.112 | 0.70 | 0.49 |
| 6 | 73.5365 | 74.2765 | −0.74 | 0.55 |
| 8 | 82.816 | 84.0835 | −1.27 | 1.61 |
| 10 | 86.5725 | 90.1365 | −3.56 | 12.70 |
| 12 | 89.0095 | 93.258 | −4.25 | 18.05 |

| | |
|---|---|
| Sum (Difference 2) = | 44.13 |
| ((Sum(Difference2)/n) + 1)0.5 | 2.55 |
| 100/(Sum(Difference\hat{ }2)/n + 1) 0.5 | 39.17 |
| F = 50 * Log[100/(Sum(Difference2)/n + 1)0.5)] = | 79.65 |

TABLE 20
Determination of similarity factor 12 according to FDA regulations
at storage conditions of 40° C./75% RH, closed, for 6 months.

| Time | Response | Response | | |
|---|---|---|---|---|
| (hrs) | (Reference product) | (Test product) | Difference | Difference 2 |
| 1 | 20.362 | 18.368 | −0.25 | 0.06 |
| 2 | 40.0745 | 32.0325 | −1.82 | 3.30 |
| 3 | 54.039 | 47.1565 | −2.72 | 7.37 |
| 4 | 67.5455 | 59.015 | 0.70 | 0.49 |
| 6 | 80.533 | 74.4095 | −0.74 | 0.55 |
| 8 | 90.545 | 84.56 | −1.27 | 1.61 |
| 10 | 94.129 | 92.4845 | −3.56 | 12.70 |

| | |
|---|---|
| Sum (Difference 2) = | 264.81 |
| ((Sum(Difference2)/n) + 1) 0.5 | 6.23 |
| 100/(Sum(Difference\hat{ }2)/n + 1) 0.5 | 16.05 |
| F = 50 * Log[100/(Sum(Difference2)/n + 1) 0.5)] = | 60.27 |

TABLE 21
Determination of similarity factor 12 according to FDA regulations
at storage conditions of 40° C./75% RH, open, 3 months.

| Time | Response | Response | | |
|---|---|---|---|---|
| (hrs) | (Reference product) | (Test product) | Difference | Difference 2 |
| 1 | 36.8125 | 17.5065 | 19.31 | 372.72 |
| 2 | 52.055 | 26.6785 | 25.38 | 643.97 |
| 3 | 61.116 | 36.719 | 24.40 | 595.21 |
| 4 | 72.8675 | 42.309 | 30.56 | 933.82 |
| 6 | 82.841 | 50.614 | 32.23 | 1038.58 |
| 8 | 87.3565 | 53.007 | 34.35 | 1179.89 |
| 10 | 91.184 | 54.9175 | 36.27 | 1315.26 |
| 12 | 91.7985 | 55.79 | 36.01 | 1296.61 |

| | |
|---|---|
| Sum (Difference 2) = | 7376.06 |
| ((Sum(Difference2)/n) + 1) 0.5 | 30.38 |
| 100/(Sum(Difference\hat{ }2)/n + 1) 0.5 | 3.29 |
| F = 50 * Log[100/(Sum(Difference2)/n + 1)0.5)] = | 25.87 |

Table 22 summarizes the results of an assay for Talin XR and Bricanyl upon storage under different accelerated stability conditions analyzed after 1, 3 and 6 month periods. The percent decrease in the assay was less than 5% for samples stored under closed 40° C./75% RH conditions (see FIG. 18). In contrast, samples stored under open conditions at 40° C./75% RH showed a high decrease of >5% (see FIG. 19). Talin XR showed also higher decrease in assay (>5%) upon storage under daylight (see Table 22). This result indicates that Talin XR and Bricanyl samples should be kept under closed conditions, i.e. protected from moisture. Talin XR should be protected from daylight.

Table 23 summarizes the appearance of related impurities upon storage of Talin XR and Bricanyl under different accelerated stability conditions analyzed after 1, 3 and 6 month periods. The percent total impurities under closed conditions was less than 0.4% (B.P. 2003 acceptance limit) for both Talin XR and Bricanyl, as shown in FIG. 20. However, under closed conditions both Talin XR and Bricanyl failed to pass the limit, as shown in FIG. 21. Thus, total impurity limit indicates that the products should be protected from moisture.

The individual impurity limit (stated to be less than 0.2% according to B.P. 2003) was less than 0.2% for Talin XR and higher than 0.2% for Bricanyl under closed 40° C./75% RH conditions (see FIGS. 22 and 3, respectively). This indicates the superiority of our formula of Talin XR over the Bricanyl formula. Thus, Talin XR passes the test while Bricanyl fail to pass this pharmacopoeial test. On the other hand, under open conditions at 40° C./75 both Talin XR and Bricanyl failed to pass the individual impurity test (see FIGS. 24 and 25).

Table 24 summarizes different tests used to compare Bricanyl and Talin XR after 6-month storage. Accordingly, Talin XR provides a more stable formula than Bricanyl under closed storage condition at a temperature of 40° C.

TABLE 22
Summarizes the assay results of Talin and Bricanyl.

| | | Product | | | | | |
|---|---|---|---|---|---|---|---|
| | | TalinXR | | | Bricanyl | | |
| Condition | period | average | CV % | % Drop | average | CV % | % Drop |
| initial | 0 | 97 | 1.9 | | 97.64 | 1.17 | 0 |
| 40/75 open | 1 month | 88.1 | | −8.9 | 95.37 | | −2.27 |
| | 3 months | 62.5 | 3.39 | −34.5 | 91.95 | 2.66 | −5.69 |
| | 6 months | | | | 86.8 | 4.24 | −10.8 |
| 40/75 closed | 1 month | 92.8 | | −4.2 | 99.8 | | 0 |
| | 3 months | 94.125 | 0.19 | −2.875 | 92.475 | 0.42 | −5.165 |
| | 6 months | 93.4 | 1.69 | −3.6 | 93.595 | 0.11 | −4.045 |
| 50 closed | 1 month | 91.7 | | −5.3 | | | |
| | 3 months | 96.69 | 0.13 | −0.31 | 98.75 | 0.93 | 0 |
| Daylight | 6 months | 89.405 | 2.3 | −7.595 | 99.03 | | 0 |

TABLE 23
Related impurities of Talin and Bricanyl.

| | | Product | | | | | | |
|---|---|---|---|---|---|---|---|---|
| | | TalinXR | | | Bricanyl | | | |
| Condition | period | imp C | unk 1.9 | unk 0.58 | imp C | unk 0.28 | unk 0.58 | unk 0.8 |
| Initial | 0 | 0.01 | | | 0 | 0.01 | 0.05 | |
| 40/75 | 1 month | 0.05 | | | 0.1 | | | 0.47 |
| open | 3 months | 0.05 | 0.36 | | 0.18 | | | 0.8 |
| | 6 months | | | | | | | |
| | 1 month | 0.01 | | | 0.02 | | | 0.1 |
| 40/75 | 3 months | 0.02 | | | 0.04 | | | 0.2 |
| closed | 6 months | 0.03 | | 0.1 | 0.07 | | | 0.3 |
| 50C | 1 month | 0.01 | | | | | | |
| closed | 3 months | 0.02 | | | | | | |
| Daylight | 6 months | 0 | 0 | | 0.14 | | 0.05 | |

TABLE 24
Summary of assay and related after 6 month storage.

| | Bricanyl | | | Talin XR | | |
|---|---|---|---|---|---|---|
| | 40/75 | 40/75 | | 40/75 | 40/75 | |
| | open | closed | Daylight | open | closed | Daylight |
| % Dec | (11%) fail | (4%) pass | (0%) pass | (>34%) fail | (3.6%) | (7.6%) |
| Assay | | | | | pass | fail |
| Related, | (0.39%, 2%) | (0.3%) fail | (0.14%) pass | (>0.36) fail | (0.1%) | (0%) |
| individual* | fail | | | | pass | pass |
| Related, | (2.4%) fail | (0.37%) | (0.19%) pass | (>0.41%) | (0.13%) | (0%) |
| total | | pass | | fail | pass | pass |
| Physical | pass | pass | fail (brownish | fail | pass | pass |
| appearance | | | discoloration) | (black spots) | | |
| Dissolution | | | | not similar | similar | similar |
| profile | | | | | | |
| similarity | | | | | | |
| Net Result | fail | fail | fail | fail | pass | fail |
| of each | | | | | | |
| condition | | | | | | |

*The highest value of individual impurity was recorded

### EXAMPLE 8

The objective of this study is the evaluation of Talin XR using a high speed tableting machine (Fette Tablet Press Machine P2100) of a SCALING UP (15 kg) Batch of Talin 5 XR tablets containing 5 mg Terbutaline sulfate.

Table 25 shows bulk density and sieve analysis of Talin 5 XR powder indicating the mixture particle size is <0.25 mm. This is useful in the case of these polymers since larger particle size was found to increase elastic recovery and decrease tablet hardness. Powder is flowable in tableting machine speed range and the tableting rate is >70,000 tablets/hr.

TABLE 25
Bulk density and sieve analysis of Talin 5 XR powder.

| Item | Result |
|---|---|
| Bulk density (g/cc) | 0.53 |
| Sieve analysis (%): | |
| 1.0 mm | 0 |
| 0.71 min | 0 |
| 0.50 min | 0 |
| 0.355 mm | 1 |
| 0.25 mm | 1 |
| 0.18 + 0.09 mm | 83 |
| 0.06 mm | 13 |
| Fine | 2 |

Table 26 shows the uniformity of Terbutaline sulfate distribution in the mixture after 10 min mixing using the V-mixer. The results indicate the close proximity in content detection between UV and HPLC methods.

TABLE 26
Content uniformity of the powder mixture of Talin XR
before compression.

| | | Terbutaline sulfate | |
|---|---|---|---|
| | | content | Terbutaline sulfate |
| | Concentration | (UV spec | content |
| Sample site | (mg/100 ml) | method) (%) | (HPLC method) (%) |
| Lower part | 7.01 | 100.38 | 101.65 |
| | 7.12 | 99.60 | 102.16 |
| Middle part | 7.06 | 100.48 | 99.69 |
| | 7.04 | 103.88 | 103.79 |
| Upper part | 7.10 | 104.65 | 106.19 |
| | 7.11 | 105.24 | 101.65 |

FIG. 26 shows a weight variation plot of 40 tablets taken randomly from the batch (as a representative sample) of Talin XR. The plot indicates that the tablets' individual weights are within the acceptance limits of BP 2003 weight variation test.

FIG. 27 shows tablet hardness (N) plot of 20 tablets (representative sample) of Talin XR. All tablet hardness fall within the proposed limits (40-100 N). The estimated friability of 20 tablets was 0.01%. This is accepted since the limit is <1%.

FIG. 28 shows content uniformity of 10 tablets (representative sample) of Talin XR indicating that these tablets are within the accepted BP 2003 limits (85-115).

FIG. 29 shows a dissolution profile of Talin XR compared to Bricanyl 5 mg tablets. The profile indicates the similarity in release with Bricanyl, as shown also in Table 27, where the similarity factor f2>50.

An assay of Talin XR tablets taken from various sites in the batch indicates the homogeneity in the results of assay, as shown in Table 28. The assay was 100% using HPLC method and 98% in case of UV-method. This variation may be related to method of analysis. The sample passes the BP 2003 test of assay.

Three impurities appear unknown 1 (relative retention time RRT 0.6), unknown 2 (RRT 0.8) and impurity C (RRT 0.76). The % related in the sample was around 0.02% for each individual impurity. The BP 2003 limit for individual impurity is 0.2%. The total impurity limit should not be more than 0.4%. Consequently, the batch passes test of impurities the BP test. As a conclusion, this formula is applicable in the large-scale production machines.

TABLE 27
Determination of similarity factor f2 according to FDA regulations
obtained according to HPLC analysis.

| Time | Response | Response | | |
|---|---|---|---|---|
| (hrs) | (Reference product) | (Test product) | Difference | Difference 2 |
| 1 | 17.14 | 24.32 | −7.18 | 51.56 |
| 2 | 27.48 | 34.75 | −7.27 | 52.82 |
| 3 | 42.42 | 48.48 | −6.05 | 36.64 |
| 4 | 53.12 | 57.13 | −4.01 | 16.04 |
| 6 | 68.95 | 70.18 | −1.23 | 1.52 |
| 8 | 78.58 | 79.43 | −0.85 | 0.73 |
| 10 | 86.75 | 84.31 | 2.45 | 5.98 |
| 12 | 91.26 | 86.60 | 4.66 | 21.72 |

| | |
|---|---|
| Sum (Difference 2) = | 187.01 |
| ((Sum(Difference2)/n) + 1) 0.5 | 4.94 |
| 100/(Sum(Difference\hat{ }2)/n + 1) 0.5 | 20.25 |
| F = 50 * Log[100/(Sum(Difference2)/n + 1)0.5)] = | 65.33 |

TABLE 28
Assay (initial) of Talin 5 XR

| | Final | Conc % | |
|---|---|---|---|
| Sample site | (mg/100 ml) | UV spec method | HPLC method |
| Sample 1 - lower | 6.92 | 100.2278 | 104.2 |
| Sample 2 - lower | 6.87 | 94.80033 | 98.9 |
| Sample 3 - lower | 6.76 | 97.78688 | 101.6 |
| Sample 1 - mid | 6.93 | 98.04861 | 99.9 |
| Sample 2 - mid | 6.85 | 96.18543 | 98.6 |
| Sample 1 - up | 6.91 | 96.05654 | 99.4 |
| Sample 2 - up | 6.98 | 100.7258 | 99.5 |
| Average (% CV) | | 97.7 (2.3) | 100.3 (2.0) |

1. A controlled-release pharmaceutical composition comprising at least Terbutaline sulfate or a derivative thereof as an active agent, and further comprising an inactive matrix, said matrix comprising a hydrophilic polysaccharide polymer mixture, said mixture comprising chitosan or a derivative thereof, and further comprising xanthan gum or a derivative thereof, wherein the ratio of xanthan gum and chitosan within said mixture is in the range from about 1:10 to about 10:1.

2. The controlled-release pharmaceutical composition according to claim 1, wherein the ratio of xanthan gum and chitosan is in the range from about 1:2 to about 2:1.

3. The controlled-release pharmaceutical composition according to claim 1, wherein the ratio of xanthan gum and chitosan is 1:1.

4. The controlled-release pharmaceutical composition according to claim 1, wherein the ratio of Terbutaline sulfate and the hydrophilic polysaccharide polymer mixture is in the range from about 1:1 to about 1:5, and preferably is 1:3.

5. The controlled-release pharmaceutical composition according to claim 1, optionally comprising at least one additional pharmaceutically acceptable filler.

6. The controlled-release pharmaceutical composition according to claim 5, wherein the pharmaceutically acceptable filler is selected from the group comprising calcium hydrogen phosphate, mannitol, Avicel PH 101, sodium bicarbonate and the like.

7. The controlled-release pharmaceutical composition according to claim 5, wherein the pharmaceutically acceptable filler is a carbon dioxide generating and/or pH controlling filler.

8. The controlled-release pharmaceutical composition according to claim 5, wherein the pharmaceutically acceptable filler is sodium bicarbonate.

9. The controlled-release pharmaceutical composition according to claim 5, wherein the percentage of the pharmaceutically acceptable filler is in the range from about 5% to about 20% by total weight, and preferably is 12.5% by total weight.

10. The controlled-release pharmaceutical composition according to claim 5, wherein the percentage of the pharmaceutically acceptable filler together with the hydrophilic polysaccharide polymer mixture is in the range from about 70 to about 90% by total weight, and preferably is 83% by total weight.

11. The controlled-release pharmaceutical composition according to claim 5, wherein the ratio of Terbutaline sulfate and the pharmaceutically acceptable filler on the one hand and the hydrophilic polysaccharide mixture on the other hand is in the range of about 1:1 to about 1:5, and preferably is 1:5.

12. The controlled-release pharmaceutical composition according to claim 1, wherein a single dosage unit of Terbutaline sulfate is about 4 to 8% by total weight.

13. A method for preparation of a controlled-release pharmaceutical composition according to any of the preceding claims comprising at least Terbutaline sulfate or a derivative thereof as an active agent, said method comprising the following steps:
(a) preparing a mixture comprising chitosan or a derivative thereof, xanthan gum or a derivative thereof, Terbutaline sulfate or a derivative thereof and optionally at least one additional pharmaceutically acceptable filler;
(b) compacting said mixture, preferably into a tablet.

14. The method according to claim 13, wherein the tablet comprises from about 5 mg to about 7.5 mg Terbutaline sulfate, and preferably comprises 5 mg Terbutaline sulfate.

15. A use of a controlled-release pharmaceutical composition according to claim 1 comprising at least Terbutaline sulfate or a derivative thereof as an active agent for the manufacture of a medicament for inducing muscle relaxation in an animal, preferably a mammal, and most preferably human.
